(19) United States
(12) Patent Application Publication
(10) Pub. No.: US 2023/0093975 A1
(43) Pub. Date: Mar. 30, 2023

(54) IMPLANTABLE MATRIX FOR TREATING CENTRAL NERVOUS SYSTEM DISORDERS

(71) Applicant: LYRA THERAPEUTICS, INC., Watertown, MA (US)

(72) Inventors: Danny Concagh, Medfield, MA (US); Changcheng You, Northbridge, MA (US); Maria Palasis, Wellesley, MA (US); Allison Gartung, Watertown, MA (US)

(21) Appl. No.: 17/567,406

(22) Filed: Jan. 3, 2022

Related U.S. Application Data
(63) Continuation-in-part of application No. PCT/US21/52334, filed on Sep. 28, 2021.

(51) Int. Cl. A61K 9/00; A61K 31/27; A61P 25/28

(52) U.S. Cl. CPC A61K 9/0043; A61K 31/27; A61K 9/0004; A61P 25/28

(57) ABSTRACT

The present invention is related to the fields of drug delivery and implantable devices. Devices, systems, and methods for use of drug delivery implants are contemplated herein. The present invention relates to implantable devices, as well as their methods of use and manufacturing. Exemplary embodiments of the present invention include fiber and sheet based implantable devices for drug delivery to bodily lumens. In particular, implantable drug delivery device compositions are contemplated for use providing consistent drug delivery over time to the central nervous system for treating associated disorders and disease. As one specific example, a CNS delivery device is contemplated for implantation in a region of the nose for providing consistent drug delivery over time to brain tissue for treating brain associated diseases, e.g. Alzheimer's disease (AD) and related dementias (AD/ADRD).

### CROSS-REFERENCE TO RELATED APPLICATIONS

The present application is a continuation in part of, and claims benefit of, as a National Entry of pending International Patent Application PCT/US21/52331 filed on Sep. 28, 2021.

### FIELD OF INVENTION

The present invention is related to the fields of drug delivery and implantable devices for treating CNS disease and disorders. In particular, implantable compositions of drug delivery device are contemplated for use providing consistent drug delivery over time to the central nervous system, e.g., brain tissue, for treating CNS associated disorders and disease. As one specific nonlimiting example, a CNS delivery device is an implant contemplated for implantation in a region of the nose for providing consistent drug delivery over time to brain for treating brain associated diseases, e.g., Alzheimer's disease (AD) and related dementias (AD/ADRD). Devices, systems, and methods for use of coated matrix scaffold implants and/or osmotic drug delivery implants, configured to fit into specific nasal cavities, are contemplated herein. The present invention relates to implantable devices, as well as their methods of use and manufacturing. Exemplary embodiments of the present invention include fiber and sheet based implantable devices for drug delivery to bodily lumens.

### BACKGROUND

There is a demand for delivery systems that can sustainably deliver drug in a site-specific manner. Various drug delivery systems have been developed to optimize the therapeutic properties of drug products rendering them more safe, effective, reliable, and reducing issues of drug non-compliance. Implantable drug delivery systems are an example of such systems available for therapeutic use. Conventional biodegradable and nonbiodegradable (or biodurable) implants are available as monolithic systems or reservoir systems. The release kinetics of drugs from these implants depend on both the solubility and diffusion coefficient of the drug in the carrier polymer, the drug load, as well as the in vivo degradation rate of the carrier polymer, in the case of a biodegradable system. To offer this type of implant with more delivery versatility, the benefits of osmotic pumps have been integrated into biodegradable and nonbiodegradable/biodurable implants to create implants that deliver drug osmotically in conjunction with other release kinetics. These systems can deliver various types of active pharmaceutical ingredients (APIs), hydrophilic/lipophilic or small molecule/biomacromolecule, at steady rates.

As one example, central nervous system (CNS)-related diseases and injuries are difficult to treat. Many of these conditions and diseases would benefit from a therapeutic delivery system that can sustainably deliver drugs to tissues in the CNS in a site-specific manner.

### SUMMARY OF THE INVENTION

The present invention is related to the fields of drug delivery and implantable devices. Devices, systems, and methods for use of drug delivery implants (including but not limited to osmotic drug delivery implants) are contemplated herein. The present invention relates to implantable devices, as well as their methods of use and manufacturing. In one embodiment, the implantable device has osmotic capabilities, i.e. delivers drug because of osmotic forces.

Exemplary embodiments of the present invention include fiber and sheet based implantable devices for drug delivery to bodily lumens. In particular, implantable drug delivery device compositions are contemplated for use providing consistent drug delivery over time to the central nervous system for treating associated disorders and disease. As one specific example, a CNS delivery device is contemplated for implantation in a region of the nose for providing consistent drug delivery over time to brain tissue for treating brain associated diseases, e.g. Alzheimer's disease (AD) and related dementias (AD/ADRD).

The present invention relates to a CNS drug delivery device comprising an active agent, e.g., therapeutic, for treating a CNS disease or disorder and an implantable bioabsorbable (matrix) mesh scaffold configured for implantation in a nasal cavity of a mammal, such as an olfactory cleft and/or middle meatus (MM) of nasal tissue for delivery of a CNS therapeutic to adjacent tissues. Examples of mesh scaffolds in general are described in: Sharma, et al. “The development of bioresorbable composite polymeric implants with high mechanical strength.” Nat Mater. January 2018; 17(1):96-103. doi:10.1038/nmat5016).

A “CNS delivery device” refers to an implant, comprising a polymer coating for use with one or more CNS active agents. In one embodiment, the implant is a scaffold. In one embodiment, the scaffold is a mesh scaffold.

In one preferred embodiment, a mesh scaffold will be configured for implantation within an olfactory cleft adjacent to nasal tissue for eluting drug into adjacent nasal tissue. In one embodiment, a coating comprising a CNS therapeutic will be configured for desired level and rate of active agent release into adjacent tissues, as described herein.

The present invention relates to implantable devices, as well as their methods of use and manufacturing. Certain embodiments may be considered a further development of the embodiments disclosed in International Patent Publication WO2018195484A1, incorporated by reference in its entirety herein.

It is not intended that the present invention be limited to the nature of a drug release kinetics of the implant. Nonetheless, in a preferred embodiment, the drug is released in a substantially linear manner over at least 12 weeks of the implantation, e.g. from the second week to the 13^{th} week. In a more preferred embodiment, the implant exhibits a zero-order release over at least 12 weeks of the implantation, e.g. from the second week to the 13th week. In one embodiment, said first and second implants are configured to release 20 to 80% of said drug during the first 12 weeks.

It is not intended that the present invention be limited to a particular structure for the implant. Nonetheless, in a preferred embodiment, at least one of said first or second implants is a braided structure. In a preferred embodiment, at least one of said first or second implants is a tubular structure. In a preferred embodiment, at least one of said first or second implants is self-expanding. In another embodiment, said implant comprises helical strands. An exemplary implant is shown in FIG. 9C.

It is not intended that the monitoring be limited to just 12 weeks. In another embodiment, said monitoring of the first patient's CNS condition is done for a period of at least 16 weeks. In yet another embodiment, said monitoring of the first patient's CNS condition is done for a period of at least 20 weeks. In yet another embodiment, said monitoring is done for a period of 24 weeks or more.

It is not intended that the present invention be limited to how the implant is loaded with drug. However, in one preferred embodiment, said first implant (and, where another implant is desired, said second implant) comprise at least one coating, said coating containing a drug and/or agent. In one preferred embodiment, said coating is a polymer coating. In a further embodiment, the drug containing coating is overlaid (at least in part) with another polymer coating or “topcoat” lacking drug. In one embodiment, the thickness of the topcoat controls the amount and/or timing of drug release.

In one embodiment, the present invention contemplates a self-expanding implantable device comprising drug. It is not intended that the present invention be limited to the amount of self-expanding. Nonetheless, in one embodiment, the devices of the present disclosure preferably expand to from 70 to 100% of their as-manufactured configuration after being crimped (e.g. crimped to a smaller shape to facilitate delivery).

In one embodiment, the present invention contemplates a scaffold that conforms to the shape of the targeted anatomy comprising: a) a scaffold comprising a plurality of polymeric strands that comprise a first polymer material; b) a coating over the scaffold that comprises a crosslinked elastomer; and c) a layer comprising said drug. In one embodiment, the device further comprises d) a topcoat over said layer comprising said drug, wherein the thickness of said topcoat is configured so that said drug is to be released substantially linearly for more than 6 weeks after placement of the scaffold. In one embodiment, the substantially linear release is after week one. In one embodiment, the polymer material comprises poly(lactide-co-glycolide). In one embodiment, the elastomer material comprises poly(lactide-co-caprolactone). In one embodiment, the elastomer material comprises poly(lactide-co-caprolactone) having molar percentage of lactide ranging from 30 to 50% and a molar percentage of caprolactone ranging from 50 to 70%. In one embodiment, the present invention contemplates delivering said scaffold to the targeted anatomy of a human, and more specifically, delivery through the nose to a location that allows for access to the olfactory and/or trigeminal nerve pathway to maximize delivery to the brain while minimizing systemic absorption. In one embodiment, the present invention contemplates bilateral delivery of first and second scaffolds, each comprising a drug, to the first and second olfactory area of a human.

In one embodiment, the present invention contemplates a combination therapy for use in a method of treating a CNS condition, comprising first and second implants, each comprising at least one coating containing a drug, wherein the first implant is configured to fit inside the nose at a targeted portion of the nasal anatomy of a patient, said drug configured to be released into a first nasal cavity for more than 12 weeks. In one embodiment, said implant is configured to exhibit a zero-order release for at least 60% of said drug. In one embodiment, said implant is configured to exhibit a zero-order release between 1 and 12 weeks. In one embodiment, said implant is configured to exhibit a zero-order release between days 20 and 55, after implantation.

The present invention contemplates in one embodiment a method of treating a central nervous system condition, comprising: a) providing an implant comprising a therapeutic compound; and b) implanting said implant at a (first) position inside the nose of a patient having a symptom of a central nervous system condition, wherein said position allows for delivery of the therapeutic compound to the brain. In one embodiment, said position is the middle meatus. In a preferred embodiment, said position is the olfactory cleft. In one embodiment, the method further comprises implanting a second implant at a second position inside the nose of said patient, wherein said first and said second positions are corresponding areas on opposite sides of the nose. In one embodiment, said implant comprises a polymer scaffold. In one embodiment, said polymer scaffold comprises fibers. In one embodiment, said implant releases said therapeutic compound by osmosis. In one embodiment, said therapeutic compound is a cholinesterase inhibitor. In one embodiment, said cholinesterase inhibitor is rivastigmine. In one embodiment, the amount of rivastigmine in the blood is less (e.g. 50%, 40%, 30%, 20% or less) when compared to blood levels of the drug after oral delivery (e.g. when the same amount of drug is used in both cases), thereby showing that delivery to the desired target is more effective through the nasal route and results in lower amounts of systemic drug. In one embodiment, the amount of rivastigmine delivered to the brain is higher (e.g. 20%, 30%, 40%, 50% or more higher) than the amount delivered to the brain (and/or CSF) after oral administration (e.g. when the same amount of drug is used in both cases). In one embodiment, the nasal route provides continuous drug dosing at a steady rate over prolonged periods with the implant compared to oral administration. In one embodiment, said patient has been diagnosed with Alzheimer's Disease. In one embodiment, the method further comprises the step c) monitoring said patient's symptom(s) for a period of time (e.g. for hours, days, weeks to months). In a preferred embodiment, symptom(s) are monitored of at least 2 weeks. In one embodiment, the symptom is reduced. In one embodiment, said patient is a human patient.

The present invention also contemplates a device, comprising an implant comprising a cholinesterase inhibitor. In one embodiment, the device is a self-expanding device. In one embodiment, the device is configured for placement in the olfactory cleft. In one embodiment, the device is configured for placement in the middle meatus. In one embodiment, the device is crimped and loaded into a delivery device, such as a delivery catheter.

In one embodiment, said implant comprises a polymer scaffold. In one embodiment, said polymer scaffold comprises fibers. In one embodiment, said implant releases said cholinesterase inhibitor by osmosis. In one embodiment, said cholinesterase inhibitor is rivastigmine.

### Definitions

Terms such as “about” and “approximately” are intended, when used with a number, to indicate a range of plus/minus 10%.

A “patient” refers to any mammal animal used for evaluation and treatment as described herein, including but not limited to mammals, such as humans, rabbits, rodents, test mammal, a mammal model for testing a device and/or drug formulation and a mammal in need of treatment for a CNS disorder.

The term “drug delivery” as used herein, relates to systems for transporting pharmaceutical compounds to a bodily system.

The term “agent” as used herein, relates to a substance that brings about a chemical, biological, or physical effect or reaction.

The term “agent” refers to active ingredients, such as active pharmaceutical ingredients (APIs), active agents, such as therapeutic agents, therapeutics and drugs.

The term “active pharmaceutical ingredient” as used herein, relates to a substance or mixture of substances, that are intended to furnish pharmacological activity or other effect in the diagnosis, cure, mitigation, treatment, or prevention of disease or to affect the structure or function of the body.

The term “drug” as used herein, relates to a pharmaceutical compound.

The term “therapeutic agent” as used herein, relates to a substance or compound (drug, protein, peptide, gene, etc.) capable of having a healing or treating effect.

The term “first pass effect” and “first-pass metabolism” refers to a phenomenon of drug metabolism whereby the concentration of a drug, specifically when administered orally, is greatly reduced before it reaches the intended target and/or tissue. First-pass metabolism includes reference to a fraction of therapeutic drug lost during the process of absorption or passage through a tissue barrier, including but not limited to a blood vessel wall, a gastrointestinal wall, a BBB, etc.

The term “substance” as used herein, relates to a physical matter.

The term “small molecule” refers to a molecule below the molecular weight of 1 kDa. The term “biomacromolecule” as used herein, relates to biomolecules having a molecular weight over 0.8 kDa.

The terms, “XTREO™” and “XTREO™ platform” and “XTREO™ matrix” and “XTREO™ technology platform” and “XTREO™ drug delivery technology platform” refers to an implant scaffold (such as that shown in FIG. 9C) comprising one or more pharmaceuticals.

A modified XTREO™ (technology) platform refers to an implantable elastomeric coated matrix comprised of biocompatible polymers coated with an elastomer, e.g. coating, and drug formulation designed for continuous release of a CNS therapeutic at a desired steady rate. As such, modifications may include configuring for implantation into a specific nasal cavity, modifications to the coating for absorbing and releasing exemplary therapeutics, at contemplated amounts, contemplated levels, over desired times as described herein.

The term “cavity” as used herein, relates to an empty space within an object, such as within a human or animal body.

The term “nasal cavity” as used herein, relates to spaces, cavities, and lumen within the nose, above and behind the nose in the middle of a face. Lumens in the nasal cavity include the superior meatus, the middle meatus, and the inferior meatus. Another nasal cavity is the “olfactory cleft.” The olfactory cleft refers to a paired orifice located in the medial and upper regions of the nasal cavity. This cleft is limited by the middle turbinate laterally, the nasal septum medially, the cribriform plate and the superior turbinate superiorly, the inferior margin of the middle turbinate inferiorly, and the anterior face of sphenoid sinus posteriorly.

As used herein, terms “sinus” and “sinus cavity” refer to cavities which include the maxillary, frontal and ethmoid sinuses, the ethmoid infundibulum and the sphenoid sinuses. The middle meatus refers to a nasal cavity that is not a sinus cavity. The ostiomeatal complex is a channel that links the frontal sinus, anterior ethmoid air cells and the maxillary sinus to the middle meatus, allowing airflow and mucociliary drainage.

The term “sinus” as used herein, relates to the paranasal sinuses, the spaces, cavities, or lumens in the cranial bones. Sinus cavities include the frontal sinus, the sphenoid sinus, the ethmoid air cells, and the maxillary sinus.

As used herein, a “condition” refers to a particular state of being. A “medical condition” also is a broad term that includes disorders, diseases, lesions, and symptoms thereof.

A medical condition may also include a specific type of area of the body, such as when using the term “CNS condition.”

As used herein, “generally tubular” includes hollow shapes of circular cross-section or non-circular cross-section (e.g., oval, etc.) and hollow shapes of constant diameter or variable diameter (e.g. of tapered diameter, such as in a hollow frustum).

The term “scaffold” as used herein, relates to a structure comprising a supporting framework. For example, the scaffold can be wrapped or rolled sheet(s) of materials (as shown in some of the figures) or a device comprising a structure of fibers, strands or filaments. In one embodiment, the scaffold is a self-expanding scaffold.

As used herein, “device,” “scaffold,” “stent”, “carrier”, “matrix”, “mesh” and “implant” may be used synonymously.

Scaffolds in accordance with certain embodiments of the present disclosure are provided with expansion and mechanical properties suitable to render the scaffolds effective for its intended purpose. Two measures of such mechanical properties that are used herein are “radial resistive force” (“RRF”) and “chronic outward force” (“COF”). RRF is the force that the scaffold applies in reaction to a crimping force, and COF is the force that the scaffold applies against a static abutting surface. In certain embodiments, the scaffolds are configured to have a relatively high RRF to be able to hold open bodily lumens, cavities, and nasal features, and the like, yet have a relatively low COF so as to avoid applying possibly injurious forces against the walls of bodily lumens, optic nerve, brain, or the like. For example, the scaffolds of the present disclosure preferably expand to from 70 to 100% of their as-manufactured configuration after being crimped. Scaffolds in accordance with certain embodiments of the present disclosure are typically tubular devices which may be of various sizes, including a variety of diameters and lengths, and which may be used for a variety of sinus applications. In the case of objects of non-circular cross-section, “diameter” denotes width.

As used herein, “strength” and “stiffness” may be used synonymously to mean the resistance of the medical scaffolds of the present disclosure to deformation by radial forces or a force applied by the scaffolds against a static abutting object. Examples of strength and stiffness measurements, as used to characterize the medical scaffolds of the present disclosure, include radial resistive force and chronic outward force, as further described herein.

In one embodiment, implantable medical devices of certain embodiments of the present disclosure are generally tubular devices, which devices are self-expanding devices in various embodiments.

Implantable medical devices of certain embodiments of the present disclosure are generally self-expanding devices. As used herein, “self-expanding” is intended to include devices that are crimped to a reduced delivery configuration for delivery into the body, and thereafter tend to expand to a larger suitable configuration once released from the delivery configuration, either without the aid of any additional expansion devices or partial aid of balloon-assisted or similarly-assisted expansion. The many scaffold embodiments of the present disclosure are self-expanding in that they are manufactured at a first diameter, subsequently reduced or “crimped” to a second, reduced diameter for placement within a delivery catheter, and self-expand towards the first diameter when extruded from the delivery catheter at an implantation site. The first diameter may be at least 10% larger than the diameter of the bodily lumen into which it is implanted in some embodiments. The scaffold may be designed to recover at least about 70%, at least about 80%, at least about 90%, up to about 100% of its manufactured, first diameter, in some embodiments.

As used herein “strands” and “filaments” may be used interchangeably and include single fiber strands and filaments (also referred to as monofilaments) and multi-fiber strands and filaments. In some embodiments, which may be used in conjunction with any of the embodiments, a braided structure may comprise opposing sets of helical strands. For example, each set of helical strands may comprise between 2 and 64 members, more typically between 8 and 32 members.

The term “implant” as used herein, relates to a device or system to be inserted into tissue, organ, or part of the body or introduced into a bodily cavity, e.g. implantation. In some preferred embodiments, an implant is a “long term” implant, such that an implant may be in contact with tissue for up to 20 weeks, up to 24 weeks, up to 30 weeks or more. In some preferred embodiments, an implant is a “Long-acting” implant, such that a patient with an implant in contact with nasal tissue may show a treatment effect, as nonlimiting examples, a patient feels improvement in at least one symptom, a patient shows an improvement of at least one symptom, (such that a symptom level is rescued), during a clinical exam that may or may not include a diagnostic, such as CT, MRI, versions thereof, etc., for up to 4 weeks, up to 8 weeks, up to 12 weeks, up to 16 weeks, up to 20 weeks, up to 24 weeks, up to 30 weeks or more.

The term “braided” as used herein, relates to a structure, such as a device, comprising one or more intertwined strands.

The term “helical” as used herein, relates to a spiral or helical shaped structure, comprising one or more strands. As used herein, “helical” and “spiral” may be used synonymously.

The term “spiral” as used herein, relates to a spiral or helical shape structure, comprising one or more strands. As used herein, “helical” and “spiral” may be used synonymously.

The term “mesh” as used herein, relates to a structure, such as a device, made out of a network of fibers.

The term “weave” as used herein, relates to a structure, such as a device, made out of interlaced fibers passing in on direction with others at a right angle to them.

The term “tubular” as used herein, relates to hollow shapes of circular cross-section or non-circular cross-section (e.g., oval, etc.) and hollow shapes of constant diameter or variable diameter (e.g. tapered diameter, such as in a hollow frustum). Both ends of the generally tubular scaffold may be open, one end may be open and the other end closed, or both ends may be closed.

The term “expandable” as used herein, relates to a structure that has the ability to expand or widen.

The term “self-expanding” as used herein, relates to the ability for a device to expand or widen after having been contracted. The term “self-expanding” is intended to include devices that are crimped to a reduced configuration for delivery into the body, and thereafter are able expand to a larger suitable configuration (i.e. larger than the crimped configuration) once released from the delivery configuration, either without the aid of any additional expansion devices or with the partial aid of balloon-assisted or similarly-assisted expansion.

The term “osmosis” as used herein, relates to passage of fluid or molecules through a semi-permeable or permeable material. A non-limiting example includes movement of a solvent across a semipermeable membrane toward a higher concentration of solute.

The term “osmotic pump” as used herein, relates to delivery systems using movement across a permeable or semi-permeable material.

The term “osmogen” as used herein, relates to agents used to enhance osmosis.

The term “permeable” as used herein, relates to a material which allows fluids or molecules to pass through.

The term “semi-permeable” as used herein, relates to a material of which at least a portion allows fluids or molecules to pass through.

The term “strands,” “filaments,” and “fibers” may be used interchangeably and include single strands, filaments, and fibers, as well as multi-fiber strands and filaments.

The term “sheet” as used herein, relates to flat devices and systems.

The term “orifice” as used herein, relates to holes or openings within devices and systems.

The term “lumen” as used herein, relates to hollow spaces within bodily systems, devices, etc.

The term “rolled” as used herein, relates to a material wrapping around a hollow space or around itself.

The term “coating” as used herein, relates to a layer. The terms “coating” and “covering” are used synonymously herein.

The term “membrane” as used herein, relates to barrier or lining. It can be a selective barrier, allowing some things to pass through but stopping others. Such things may be molecules, ions, or other small particles.

The term “biodegradable” as used herein, relates to the ability to degrade in a bodily system. The term “bioresorbable” as used herein, relates to the ability to degrade in a bodily system. As used herein, “biodegradable” and “bioresorbable” may be used synonymously.

The terms “nonbiodegradable” and “biodurable” as used herein, relate to the ability to not degrade in a bodily system. As used herein, “nonbiodegradable” and “biodurable” may be used synonymously.

The term “aiding agent” as used herein, relates to substances which may be added to a system to aid its use.

The term “wicking agent” as used herein, relates to substances which may aid in the ability to absorb or draw in fluid or molecules.

The term “swelling agent” as used herein, relates to relates substances which may aid in the ability for a material to swell or enlarge.

The term “surfactant” as used herein, relates to substances which tends to reduce the surface tension of a fluid in which it is added.

The term “solubilizing agent” as used herein, relates to a substance which may increase solubility of one substance in another.

The term “permeability enhancer” as used herein relates to a substance which serves to facilitate the permeability of the drug into tissues or across tissue boundaries, such as the blood brain barrier for example.

The term “polymer” as used herein, relates to a substance which has a molecular structure consisting partly or entirely of a large number of units bonded together.

The term “hydrophilic” as used herein, relates to an ability to at least partially dissolve or be wetted by water. A hydrophilic molecule or portion of a molecule is one whose interactions with water and other polar substances are more thermodynamically favorable than their interactions with oil or other hydrophobic solvents. They are typically charge-polarized and capable of hydrogen bonding.

The term “lipophilic” as used herein, relates to an ability to at least partially repel water, or relates to an ability to at least partially dissolve in lipids or fats. As used herein, “lipophilic” and “hydrophobic” may be used synonymously.

The terms “mucosal tissue” and “mucosal surface” are meant to indicate the surface areas that comprise the mucosa. The mucosa is characterized by the presence of a semipermeable epithelial barrier. Mucosal tissue surfaces are characterized by the presence of an overlying mucosal fluid (making them typically a wet surface), for example fluids such as saliva, tears, nasal, gastric, cervical and bronchial mucus. Thus, such surfaces are found in the eyes, nose, gut, and lung. Additional mucosal surfaces are found in the oral cavity (e.g. the mouth), pharynx, tonsils, urethra, and vagina.

The term “chronic” as used herein, relates to persisting or recurring illness or symptoms.

The “core-shell structure” as used herein, relates to a structure comprising multiple layers or “shells,” wherein the innermost layer may be called a “core.”

### BRIEF DESCRIPTION OF DRAWINGS

Some exemplary embodiments are illustrated in referenced FIGs. It is intended that the embodiments and FIGs. disclosed herein are to be considered illustrative rather than restrictive.

The file of this patent contains at least one drawing executed in color. Copies of this patent with color drawings will be provided by the Patent and Trademark Office upon request and payment of the necessary fee.

FIG. 1 shows a schematic illustration of a self-expandable implant comprising osmotic drug delivery fibers (100) either not comprising orifices or comprising orifices under an opaque coating.

FIG. 2 shows a schematic illustration of an osmotic drug delivery fiber embodiment comprising one or more delivery orifices (200), a semi-permeable polymer membrane (201), an API (202) and an osmogen (203).

FIG. 3 shows a schematic illustration of an osmotic drug delivery fiber embodiment comprising one or more delivery orifices (300), a semi-permeable polymer membrane (301), an API (302), a polymer fiber core (303) and an osmogen (304).

FIG. 4 shows a schematic illustration of a spiral scaffold embodiment for osmotic drug delivery comprising a spiral scaffold (400) with a delivery orifice (401), with an expanded end-view showing the semi-permeable polymer membrane (402), an API (403), and an osmogen (404).

FIG. 5 shows a schematic illustration of a rolled osmotic drug delivery sheet embodiment comprising one or more delivery orifices (500), a semi-permeable polymer membrane (501), an API (502) and an osmogen (503).

FIG. 6 shows a schematic illustration of a self-expandable implant embodiment comprising osmotic drug delivery fibers (600) comprising orifices (601).

FIG. 7 shows schematic illustrations of exemplary pathways of agents entering nasal areas as part of exemplary pathways for agents applied to and eluted from devices in nasal areas, including from devices implanted in nasal cavity structures, e.g., olfactory cleft (e.g., area next to olfactory epithelium near and next to the olfactory bulb), and/or from a device placed in the middle meatus. Further illustrated are exemplary areas in the brain and spinal cord, spinal cord fluid, that may receive therapeutic agents from delivery devices implanted in the nose, i.e. nose-to-brain delivery. Selvaraj et al. Artificial cells, Nanomedicine, and Biotechnology. 2018. 46(8):2088-2095.

FIG. 8 shows a Computed Tomography (CT) of nasal cavity structures (above) including the olfactory cleft, ethmoidal roof of ethmoidal sinuses, ethmoidal lateral lamina and conchal lamina (nasal turbinates), middle turbinate and a schematic illustration below depicting exemplary locations of drug delivery device implantation in the olfactory cleft and middle meatus. The lower right image shows an example of a delivery device configured to self-expand and fit into a MM for drug delivery.

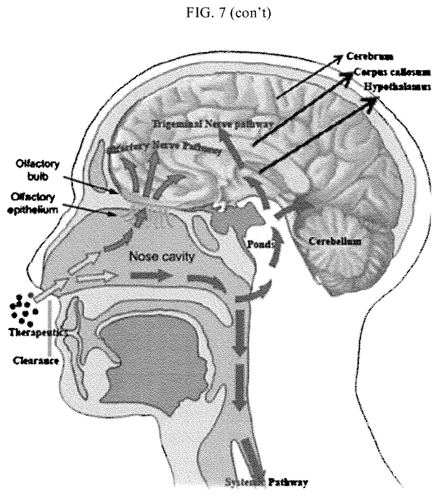

FIG. 9A is an illustration showing anatomical labeling, as a representation of a MRI image.

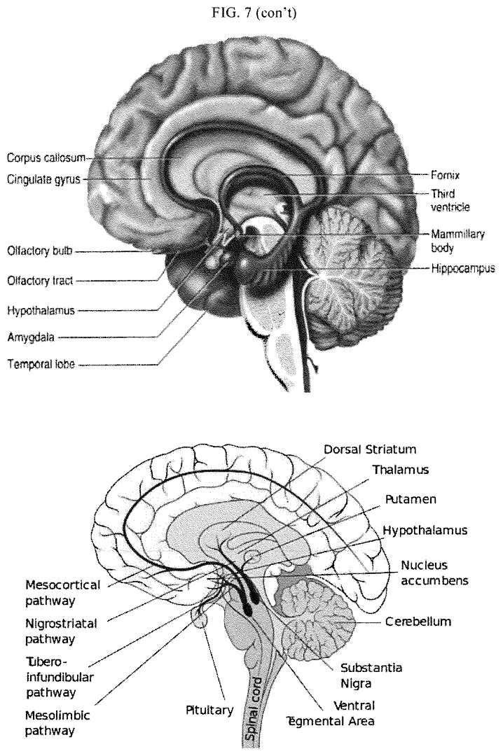

FIG. 9B illustrates, for reference, a magnified endoscopic view looking into a left (in relation to the patient) nostril, showing a septum (S), a middle turbinate (MT) and a middle meatus (MM).

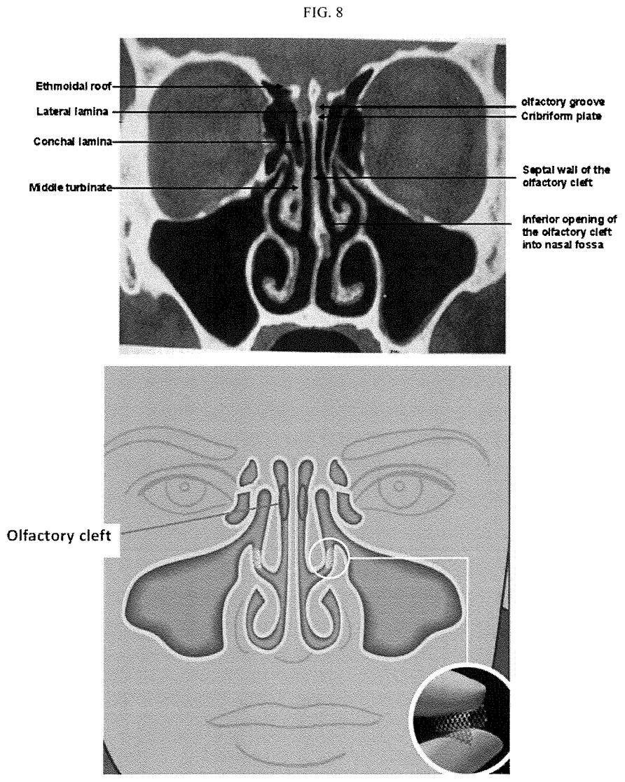

FIG. 9C illustrates a partial deployment of the scaffold (A) from the end of the applicator sheath (B), outside of nose, merely for illustrative purposes. A matrix scaffold self-expands from a constrained state when deployed from an applicator.

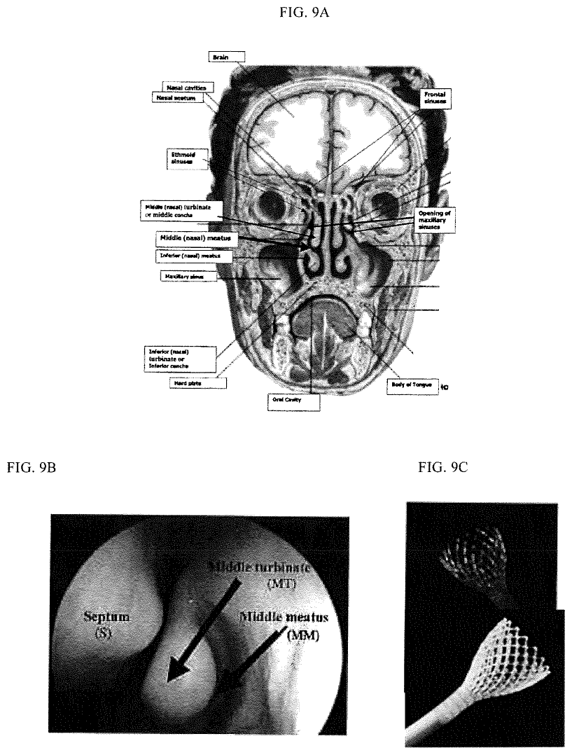

FIG. 10 shows an exemplary schematic illustration of linear drug release over time into the blood stream from a self-expandable implant in any of epithelium lining nasal cavities, lining of sinus cavities and lining olfactory cleft.

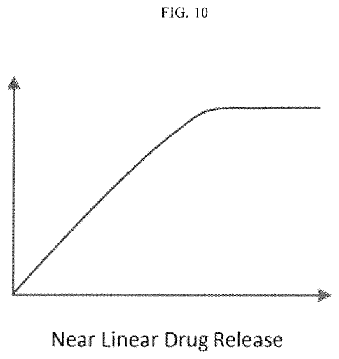

FIG. 11 shows an exemplary schematic illustration of estimated comparative delivery of rivastigmine to plasma vs. brain (e.g., exposure ratio) (above) that is altered with a CNS mesh delivery device of the present invention (below). In other words, this is a contemplated comparison of plasma levels up to and over 24 and 48 hours. However it is not meant to limit sampling timepoints to these times, e.g. timepoints may be 12, 36, 64, 72, hours or more of standard oral/subcutaneous (SC) delivery of a rivastigmine dose (above), to a CNS mesh delivery device of the present invention (below), i.e., showing less drug in plasma, more drug in brain. Top graph=standard rivastigmine administration: Plasma (dotted line) indicates rapid drug absorption and exposure that declines overtime as rivastigmine is metabolized. Brain (bars) shows contemplated detectable levels of rivastigmine. Repeated dosing (daily) creates this pattern—exposure in the plasma, absorption into the brain, plasma drug level falls, so the patient needs another dose.

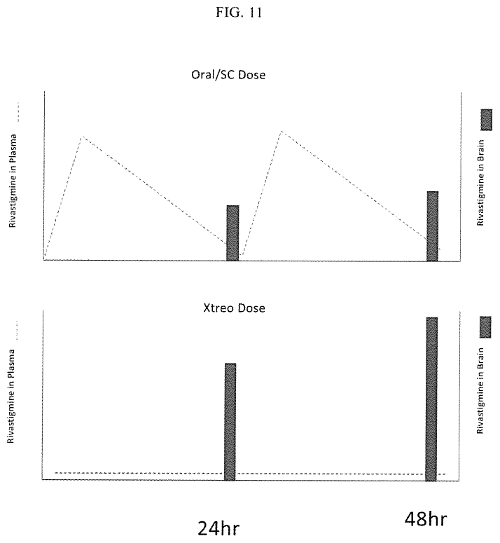

Bottom graph=a CNS mesh delivery device comprising rivastigmine. Following implantation of the mesh delivery device into a nasal cavity, there is minimal drug exposure in the plasma, and more drug released into the brain than with administration of rivastigmine oral/subcutaneous (SC) delivery. Administration of rivastigmine into nasal tissue using a CNS mesh delivery device as described herein, is contemplated to provide benefits to the patient including reduce systemic side effects, optimize getting more drug to target site (brain) quickly, improve patient outcomes, and eliminate issues with patient compliance.

### DESCRIPTION OF THE INVENTION

The present invention is related to the fields of drug delivery and implantable devices. Devices, systems, and methods for use of drug delivery implants are contemplated herein. The present invention relates to implantable devices, as well as their methods of use and manufacturing. Exemplary embodiments of the present invention include fiber and sheet based implantable devices for drug delivery to bodily lumens. In particular, implantable drug delivery device compositions are contemplated for use providing consistent drug delivery over time delivered to the central nervous system for treating associated disorders and disease. As one specific example, a CNS delivery device (e.g., comprising a therapeutic intended for relief of symptoms, or to slow onset of increased severity of symptoms, or to slow or prevent onset of a symptom) is contemplated for implantation in a region of the nose for providing consistent drug delivery over time to brain tissue for treating brain associated diseases, e.g. Alzheimer's disease (AD) and related dementias (AD/ADRD).

One major problem with delivering therapeutic molecules to the brain from circulation is the presence of an intact blood-brain-barrier (BBB). A healthy BBB excludes, or slows the entry of, many types of agents and therapeutics from entering brain tissue. Further, mere passage of BBB permeable therapeutic through the BBB may trigger first passage metabolism effects. Therefore, at least one major advantage of using an implantable CNS agent delivery device implanted in nasal/sinus/olfactory cleft openings, is for delivery of a therapeutic into the brain, and in surrounding tissues, by bypassing the BBB. Similarly, there is a blood-cerebrospinal fluid (CSF) barrier (BCSFB) comprising choroid plexus epithelial cells. Choroid plexus epithelial cells also having a secretory function, e.g., producing cerebrospinal fluid (CSF). Thus, choroid plexus epithelial cells may be a therapeutic target for a device as described herein. Therefore, another major advantage of using an implantable CNS agent delivery device, implanted in any one or more of nasal, sinus, olfactory cleft openings, is for delivery to the brain by bypassing the BCSFB.

Thus, one benefit of using devices for intranasal drug delivery to the brain is bypassing the BBB and/or BCSFB for allowing agent/therapeutic agent delivery to brain tissue, e.g., through sub-perineural epithelial and inter-axonal spaces. Exemplary pathways for therapeutic drug delivery include but are not limited to Olfactory Nerve Pathway, Trigeminal Nerve Pathway, and Systemic Absorption (Blood). Intravascular pathway: The nasal mucosa is highly vascular, allowing intranasally administered drugs to be efficiently absorbed into the vessels within the nasal mucosa and delivered into systemic circulation. CSF or lymphatics pathways: The connections between the subarachnoid space, the perineurial spaces around olfactory nerves, and nasal lymphatics also provide potential pathways for intranasally administered agents to reach the CNS, though the relative contribution of these routes remains to be elucidated.

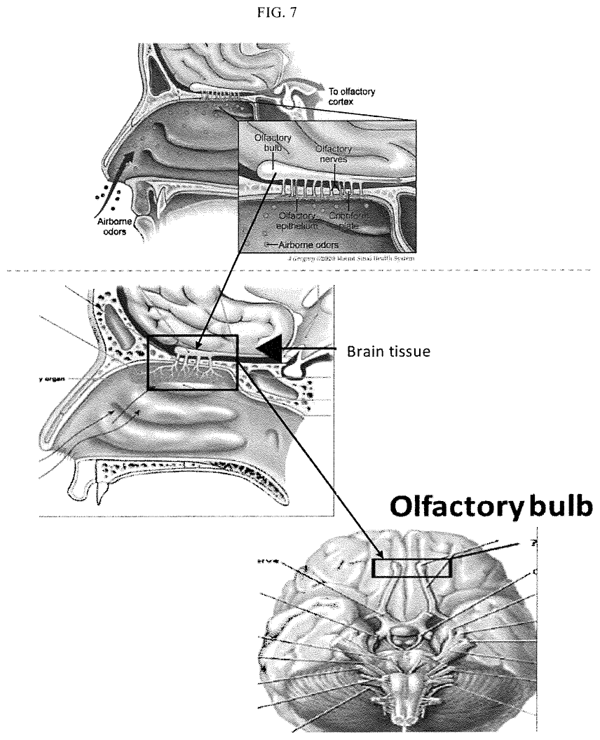

The olfactory cleft has direct fluid and cellular connections to parts of the brain, including but not limited to, thalamus, neocortex limbic system (amygdala and hippocampus) and vegetative nuclei of the hypothalamus. See, FIG. 7 and exemplary descriptions below, for examples of brain tissue contemplated to receive agents eluted into and through nasal tissues. Whereas exact mechanisms underlying intranasal drug delivery to the CNS are unknown, the following are merely nonlimiting examples of how agents may migrate from the nose to the brain, and other parts of the CNS.

As therapeutic agents have evolved to treat central nervous system (CNS) afflictions, the presence of an intact blood brain barrier (BBB) has prevented the use of many of these drugs for treating neurodegenerative diseases, such as Alzheimer's, Parkinson's, tumors, and other CNS diseases. The BBB blocks entry of many traditional and newly discovered drugs inside the brain that can protect neurons; promote nerve repair; and cure, curtail, and treat many untreatable CNS diseases. This problem may be resolved by the use of the intranasal olfactory mucosa to deliver therapeutic agents to the CNS bypassing the BBB. This simple, rapid delivery route is ideal over any other routes of delivery due to connections and transportation routes between the nasal olfactory mucosa, olfactory nerves, olfactory bulb, subarachnoid space, cerebrospinal fluid (CSF), and CNS. The following will briefly describe how therapeutic and non-therapeutic agents, can reach the brain, bypassing the BBB based on nasal and nasal olfactory mucosal routes and associated CNS connections that allow transportation directly from nasal tissue into the CNS. The olfactory mucosal region which receives agents from a device include anterior ethmoidal nerves, a branch of ophthalmic division of the trigeminal nerve, and a small fasciculus branch from the sphenopalatine ganglion whereafter therapeutic agents spread to areas of the brain including the temporal lobe, hypothalamus, thalamus, amygdala, entorhinal cortex, hippocampus, prefrontal cortex, etc. The ethmoid sinus, another target location for an implant, is situated between the orbit and the nose. The ethmoid sinus and orbit share the thin medial orbital wall, the lamina papyracea. The anterior ethmoid sinus drains into the middle meatus, and the posterior ethmoids drain into the sphenoethmoidal recess.

Therapeutic agents absorbed from the blood vessels of the olfactory and nasal mucosa may also reach the choroid plexus, which allows therapeutic agents to permeate to the ventricle, central canal of the spinal cord, then to CSF and then to neuropile close to the ependymal lining from systemic absorption through the respiratory and nasal mucosa. As another example, from the intercellular route of the olfactory mucosa, therapeutic agents are transported to sub-perineural epithelial and inter-axonal spaces of the olfactory nerves. The therapeutic agents may spread around the olfactory bulb's subarachnoid space CSF through the olfactory nerves entering through the cribriform plate of the ethmoid bone. As another example, absorbed therapeutic agents are transported to olfactory bulb subarachnoid space CSF, therapeutic agents are transported to the CSF in the subarachnoid space, specifically to the suprachiasmatic and interpeduncular CSF cisterns then to neuropile through the CNS Virchow-Robin space and blood vessels' paravascular routes. As another example, nasally absorbed therapeutic agents are spread to the temporal lobe, hypothalamus, thalamus, amygdala, entorhinal cortex, hippocampus, prefrontal cortex, and such from this subarachnoid space and CSF cisterns. As one example of delivery to the brain, devices eluting therapeutic agents into nasal tissue may treat Parkinson's, Alzheimer's, and other neurodegenerative diseases may use these main transportation routes into the brain bypassing the BBB.

As a further example, absorbed therapeutic agents within the CSF pool around the olfactory bulb and brain, may spread to the subarachnoid space around the spinal cord due to CSF circulation to then become distributed into the neuropile and neurons of the spinal cord through the Virchow-Robin space and parascular glymphatic routes. Thus, therapeutic agents from CSF delivered through olfactory nerves may spread to the brain structures and neuropile through the CSF and subarachnoid space, the Virchow-Robin space, paravascular routes, and glymphatic routs deep into the brain and spinal cord to the site of pathology for healing.

Another example of routes agents may take from nasally placed CNS treatment devices, is along trigeminal nerves. The trigeminal nerves run from the nasal mucosa to the trigeminal ganglion and from there one branch leads to the pons and other caudal brain structures. There is a second branch that leads from the trigeminal ganglion up to the cribriform plate which then passes through the foramina of the cribriform plate along with the olfactory axon bundles into the subarachnoid space and into the brain and CSF.

After drugs travel extracellularly along the olfactory and trigeminal neural pathways to the brain and flow both past and into the olfactory bulb, the drug does not have to enter the olfactory bulb and then leave it to reach other brain regions. Rather it may be like a river of drug that flows past the olfactory bulb and then onto other brain regions such as the hippocampus. Because the drug flows past the olfactory bulb first, the olfactory bulb will likely have a high concentration of drug entering it.

Thus, in preferred embodiments, a patient diagnosed with one or more of a CNS disorder, may improve, and/or a patient may feel relief, after a treatment using a continuous CNS drug delivery device described herein.

### I. Nose-to-Brain Drug Delivery

In one embodiment of a CNS agent delivery device, a nose-to-brain delivery device is contemplated for use. In some embodiments, an implantable mesh scaffold device is configured and used for continuous delivery of an agent from where the device was implanted in the nose for drug delivery to the brain for treating CNS disorders. In some embodiments, an implantable CNS agent delivery device is placed in the olfactory cleft to target an olfactory nerve pathway. In some embodiments, an implantable CNS agent delivery device is placed in the olfactory cleft to target olfactory nerve pathway for delivering an agent to the brain. In some embodiments, an implantable CNS agent delivery device is placed in the middle meatus to target trigeminal and olfactory nerve pathway. In some embodiments, an implantable CNS agent delivery device is placed into the maxillary sinus via dorsal maxillary osteotomy. In some embodiments, an implantable CNS agent delivery device is placed in the middle meatus to target trigeminal and olfactory nerve pathway for delivering an agent to the brain. See exemplary delivery pathways as described herein and in the figures. Drug delivery is not limited to examples of delivery routes described herein. In some embodiments, delivery of drug begins within minutes of implantation of a device, as described herein. Such a device is contemplated to provide a continuous drug treatment lasting for days, weeks and months, as described herein.

In some embodiments, use of a CNS delivery device as described herein, results in fewer side effects in peripheral parts of the body. In some embodiments, use of a CNS delivery device as described herein, results in at least one benefit to a patient than when compared to other routes of delivery. Examples of a benefit include an improvement in one or more of memory, motor skills, etc., that is a symptom associated with the patient's CNS disease. In some embodiments, use of a CNS delivery device as described herein, results in faster relief of at least one symptom than when compared to other routes of delivery.

### II. Central Nervous System Diseases and Treatments

The central nervous system (CNS) comprises primarily the brain and spinal cord, and further includes eyes (optic neurons and associated neurons, and sensory neurons such as rods and cones), ears, sensory organs of taste, sensory organs of smell, and sensory receptors located in the skin, joints, muscles, and other parts of the body. CNS components, tissues, cells, etc., can be damaged by any one or more of the following including: trauma, infections, degeneration, structural defects (genetic and/or somatic), tumors, blood flow disruption (including abnormal vascularization, such as in arteries, veins and/or capillaries) and from having autoimmune disorders.

Such that, disorders of the nervous system may involve the following: vascular disorders, including but not limited to damage from a stroke, associated with tumors and cancer; transient ischemic attack (TIA); subarachnoid hemorrhage; subdural hemorrhage and hematoma;

extradural hemorrhage; infections, such as meningitis, encephalitis, polio, COVID-19; epidural abscess; structural disorders, such as brain or spinal cord injury; Bell's palsy; cervical spondylosis; carpal tunnel syndrome; brain or spinal cord tumors, peripheral neuropathy; Guillain-Barré syndrome; functional disorders, such as headache, epilepsy, dizziness, and neuralgia; degeneration, such as Parkinson disease, multiple sclerosis, amyotrophic lateral sclerosis (ALS), Huntington chorea, and Alzheimer disease. Additional CNS brain disorders include but are not limited to: psychiatric disorders, e.g., schizophrenia whose symptoms include brain abnormalities such as shrinkage in brain, or brain circuitry dysfunction, such as within the brain and the blood-brain-barrier, bipolar and related genetic diseases, abnormal functioning of neurotransmitters such as dopamine, etc., to name a few.

In one embodiment, a CNS delivery device as described herein, is contemplated for treating a patient having symptoms and/or diagnosed with schizophrenia. In one embodiment, a CNS delivery device as described herein, is contemplated for treating a patient having symptoms including but not limited to hallucinations. In one embodiment, a CNS delivery device as described herein, is contemplated for treating a patient having symptoms and/or diagnosed with brain injuries. In one embodiment, a CNS delivery device as described herein, is contemplated for treating a patient having symptoms and/or diagnosed with impulse control disorders. In one embodiment, a CNS delivery device as described herein, is contemplated for treating a patient having symptoms and/or diagnosed with a major depressive disorder. In one embodiment, a CNS delivery device as described herein, is contemplated for treating a patient having symptoms, e.g., delusional symptoms. In one embodiment, a CNS delivery device as described herein, is contemplated for treating a patient having symptoms and/or diagnosed with bipolar disorder.

Examples of therapeutics, including but not limited to drugs (such as drugs in use for oral or patch administration), large molecular weight drugs, biomacromolecules, small molecules, antibodies, peptides, proteins, nucleic acids, DNA, RNA, siRNA, including but not limited to encapsulated therapeutics, such as lipid encapsulated, coated molecules, etc.

As one example, Bevacizumab antibody (Avastin, Mvasi, Zirabev) (for treating tumors) targets vascular endothelial growth factor (VEGF), a protein that helps tumors form new blood vessels (a process referred to as angiogenesis), treating some types of gliomas (such as fast-growing ones such as glioblastomas) that typically regrow after initial treatment, and for treating recurrent meningiomas. For some brain tumors, drugs are administered directly into the cerebrospinal fluid (CSF, the fluid that bathes the brain and spinal cord), either in the brain or into the spinal canal below the spinal cord. Typically, a thin tube known as a ventricular access catheter may be inserted through a small hole drilled in the skull and into a ventricle of the brain during a minor operation. Another advantage of using a delivery device as described herein, is for treating a patient without surgery, without drilling a hole into the skull.

A. Treating Brain and Spinal Cord Tumors in Adults

In some embodiments, a CNS delivery device of the present inventions may be used in conjunction with other types of therapeutic treatments, oral, patch, intravenous, intravenous (IV) infusion, etc., including but not limited to drugs, small molecules, antibodies, peptides, etc.

B. Chemotherapy for Adult Brain and Spinal Cord Tumors

Some of the chemo drugs, that may be used alone or in combinations or used sequentially, e.g., for treating brain and spinal cord tumors, contemplated for use in a CNS delivery device as described herein, include but are not limited to: Carboplatin, Carmustine (BCNU), Cisplatin, Cyclophosphamide, Etoposide, Irinotecan, Lomustine (CCNU), Methotrexate, Procarbazine, Temozolomide, Vincristine, Paclitaxel, etc.

In some embodiments, a CNS delivery device of the present inventions may be implanted before, during or following another type of therapeutic (e.g., chemotherapy treatment). As one example, use of a contemplated CNS delivery device comprising Bevacizumab (or active portion thereof) may lower the dose of a steroid drug, e.g., dexamethasone, administrated orally to help reduce swelling in the brain, which is especially important for patients sensitive to steroid side effects. Examples of common side effects of oral administration of dexamethasone that may be reduced or avoided as additional benefits of using a CNS drug delivery device as described herein, include high blood pressure, tiredness, bleeding, low white blood cell counts, headaches, mouth sores, loss of appetite, and diarrhea. Less common but possibly serious side effects include blood clots, internal bleeding, heart problems, and holes (perforations) in the intestines. This drug may also slow wound healing, so usually it is not administered within a few weeks of surgery. In some embodiments, use of a CNS delivery device may be lowering severity of side effects, or avoid the use of dexamethasone altogether. In some embodiments, a CNS drug delivery device may comprise dexamethasone. In some embodiments, a CNS drug delivery device comprising dexamethasone may be implanted after a surgical procedure.

C. Central Nervous System (CNS)

In one embodiment, the patient has symptoms of a CNS disorder. In one embodiment, the patient has symptoms of a neurodegenerative disease. In one embodiment, the neurodegenerative disease is ALS. In one embodiment, the CNS disorder is Alzheimer's disease. Alzheimer's is a type of dementia that causes problems with memory, thinking and behavior. Symptoms usually develop slowly and get worse over time, becoming severe enough to interfere with daily tasks.

D. Mild Cognitive Impairment (MCI)

Mild cognitive impairment (MCI) is a stage in decline of brain function between the expected cognitive decline of normal aging and the more serious decline of dementia or other brain disorder. It's characterized by problems with memory, language, thinking or judgment. Mild cognitive impairment may be a sign of a patient's risk of later developing dementia caused by Alzheimer's disease or other neurological condition or neurological degenerative condition.

Thus, in some embodiments, a patient diagnosed with or at risk of MCI will be treated with a CNS agent delivery device as described herein. In some embodiments, a patient diagnosed with MCI at risk of developing a neurological degenerative condition, will be treated with a CNS agent delivery device as described herein. In some embodiments, such treatment is provided in order to prevent and/or remediate brain biochemical and/or neurophysiological changes caused by neurodegenerative diseases, including but not limited to age-related sensory dysfunction, motor dysfunction, or age-related decrements in balance and postural control, gait performance, and mobility.

In one embodiment, a patient diagnosed for one or more of a MCI, may remain stable or improve (reversal of symptoms) and/or a patient may feel relief after treatments using a CNS drug delivery device described herein.

E. Neurodegenerative Diseases and Neuroinflammation

In one embodiment, a patient having a neurodegenerative disorder may be treated with a CNS drug delivery device described herein.

Neurodegenerative diseases represent a significant proportion of diseases burden and affect up to one billion people globally. Inflammatory responses in the brain have been found to induce the pathogenesis of multiple diseases such as Alzheimer's disease (AD), Parkinson's disease (PD), Multiple sclerosis (MS), etc. Thus, pathways of inflammation have been the aim of therapeutics in such diseases. Even after significant advancements on the study of such pathologies, there is still no treatment that can cure such degenerative diseases.

In one embodiment, a patient having, or suspected of having, inflammation within the CNS, e.g., brain, may be treated with a CNS drug delivery device described herein.

Neuroinflammation is specifically implicated in PD, Alzheimer's, Amyotrophic lateral sclerosis (ALS), traumatic brain injury and other diseases and conditions. In some embodiments, cellular secretions are contemplated for use as biomarkers (e.g. soluble markers released by the cells that would indicate the presence, extent or nature of the neuroinflammation). Further, diseases related to inflammation in the CNS, such as caused by infections and epileptogenesis (referring to the gradual process by which a normal brain develops epilepsy) is associated with subtle neuronal damage, gliosis, and microgliosis, with a strong, and persistent inflammatory state in the microenvironment of CNS neural tissue.

Each individual human patient may experience symptoms differently. Generally, symptoms of disorders of the nervous system may improve and/or a patient feels relief of one or more symptoms after a treatment using a CNS drug delivery implant described herein, where symptoms may include one or more of: persistent or sudden onset of headaches; headaches that change or is different; loss of feeling or tingling; weakness or loss of muscle strength; loss of sight or double vision; memory loss; Impaired mental ability; lack of coordination; muscle rigidity; tremors and seizures; back pain which radiates to the feet, toes, or other parts of the body; muscle wasting and slurred speech; new language impairment (expression or comprehension). The symptoms of a nervous system disorder may look like other medical conditions or problems so a healthcare provider should be included in diagnosis and monitoring of symptoms before and/or after treatment using a CNS drug delivery implant as described herein.

### III. Treating CNS Diseases and Disorders

A. Alzheimer's Disease and Related Dementias (AD/ADRD) Treatments

Alzheimer's disease (AD) and related dementias (AD/ADRD) are debilitating conditions that impair memory, thought processes, and functioning, primarily among older adults. Patients with AD/ADRD may require significant amounts of health care and intensive long-term services and supports—including, but not limited to, management of chronic conditions, help taking medications, round-the-clock supervision and care, or assistance with personal care activities, such as eating, bathing, and dressing. In the United States, AD/ADRD affects as many as 5 million people. Roughly 13.2 million older Americans are projected to have AD/ADRD by 2050.

Alzheimer's disease (AD), the most common cause of dementia, is a progressive neurodegenerative disorder that affects approximately 10% of people aged 65 or older and 50% of people over the age of 85. The healthcare costs of a person with AD average $341,000 between diagnosis and death, with the majority of these costs borne by families. Consistent with the high individual price of AD, AD-related healthcare costs in the US are projected to exceed $1 trillion by 2050. While there is no cure for AD, two classes of drugs: cholinesterase inhibitors and N-methyl-D-aspartate (NMDA)-receptor antagonists are widely used to treat the cognitive symptoms of AD. Therefore, in some embodiments for treating cognitive symptoms of CNS disorders, therapeutic drugs delivered with devices as describe herein are cholinesterase inhibitors and N-methyl-D-aspartate (NMDA)-receptor antagonists, and monoclonal antibodies, example, Aducanumab (monoclonal antibody) which is also approved to treat AD.

TABLE 1
Alzheimer’s Disease Treatments FDA-approved drugs currently administrated
orally, by patch or i.v., that may find use in CNS delivery devices as described herein.

| | | |
|---|---|---|
| Name | | |
| (Generic/Brand) | Approved for | Side effects |

May delay clinical decline

| | | |
|---|---|---|
| Aducanumab | Alzheimer's disease | ARIA, headache and fall |
| Aduhelm ™ | | |

Treats cognitive symptoms (memory and thinking)

| | | |
|---|---|---|
| Donepezil | Mild to severe dementia | Nausea, vomiting, loss of |
| Aricept [symbol] | due to Alzheimer’s | appetite, muscle cramps |
| | | and increased frequency of |
| | | bowel movements. |
| Galantamine | Mild to moderate dementia | Nausea, vomiting, loss of |
| Razadyne [symbol] | due to Alzheimer’s | appetite and increased |
| | | frequency of bowel |
| | | movements. |
| Rivastigmine | Mild to moderate dementia | Nausea, vomiting, loss of |
| Exelon [symbol] | due to Alzheimer's or | appetite and increased |
| | Parkinson’s | frequency of bowel |
| | | movements. |
| Memantine | Moderate to severe | Headache, constipation, |
| Namenda [symbol] | dementia due to | confusion and dizziness. |
| | Alzheimer's | |
| Memantine + Donepezil | Moderate to severe | Nausea, vomiting, loss of |
| Namzaric [symbol] | dementia due to | appetite, increased |
| | Alzheimer’s | frequency of bowel |
| | | movements, headache, |
| | | constipation, confusion |
| | | and dizziness. |

[symbol]

indicates data missing or illegible when filed

B. Parkinson's Disease and Treatments

Parkinson's Disease (PD) refers to a progressive neurodegenerative disease, often lethal, where dopaminergic (DA) neurons are abnormal and degenerate over time. A clinical pathology in humans is the presence of Lewy Body formation, consisting of abnormal aggregates of α-synuclein (alpha-Syn), a protein expressed in healthy and diseased states. Triggers of this early pathology are still unclear (Phosphorylation of α-synuclein is involved). Current hypotheses for pathogenesis include: intestine-originated, neuroinfection-driven, genetic involvement, prion-like disease etc.

Parkinson's disease (PD) is the second most common degenerative neurological disorder after Alzheimer's disease. Overall, as many as 1 million Americans are living with PD, and approximately 60,000 Americans are diagnosed with PD each year. There is no standard treatment for Parkinson's disease (PD). Loss of substantia nigra (SN) neurons causes Parkinson's disease. Some of the remaining neurons in PD contain insoluble cytoplasmic protein aggregates (Lewy Bodies) that are made of aggregated alpha-synuclein.

In Parkinson's Disease (PD) and related synucleinopathies, the accumulation of alpha-synuclein (αSyn) plays a role in disease pathogenesis. Pathological assessment of post-mortem brains from PD patients has demonstrated abnormal inclusions, enriched in misfolded and aggregated forms of αSyn, including fibrils. These findings, combined with a wealth of experimental data, support the hypothesis for a role of αSyn aggregation in the formation of the Lewy bodies (LBs) and, therefore in the pathogenesis of synucleinopathies. Recently, αSyn was

Treats non-cognitive symptoms (behavioral and psychological)

| Name | | |
|---|---|---|
| (Generic/Brand) | Approved for | Side effects |
| Suvorexant | Insomnia in people living | Impaired alertness and |
| Belsomra [symbol] | with mild to moderate | motor coordination, |
| | Alzheimer’s disease | worsening of depression or |
| | | suicidal thinking, complex |
| | | sleep behaviors, sleep |
| | | paralysis, compromised |
| | | respiratory function. |

[symbol]

indicates data missing or illegible when filed

identified in body fluids, such as blood and cerebrospinal fluid, and was postulated to be also produced by peripheral tissues. However, the ability of αSyn to cross the blood-brain barrier (BBB) in either direction and its potential contribution to the endothelial dysfunction described in patients with PD, remained unclear.

A pathological examination of a healthy patient reveals typical pigmented DA neurons in the SN; in contrast, loss of SN neurons leads to pigment disappearance in the PD brain. Most of the SN neurons are lost in PD during neuronal degeneration. Some of the remaining neurons in PD contain insoluble cytoplasmic protein aggregates (Lewy Bodies) that are made of aggregated alpha-synuclein and other proteins. In some embodiments, a CNS delivery device as described herein, may be used to prevent, delay onset, reduce at least one symptom of PD, and the like. In some embodiments, use of inventive devices may produce faster and more beneficial results than when treatments are administered orally or in non-inventive device routes of administration.

### IV. Examples of Drugs and Exemplary Delivery Devices

Cholinesterase inhibitors treat AD symptoms by targeting a deficit in cholinergic neurotransmission observed in an AD-impacted brain, e.g., as in a patient exhibiting cognitive symptoms typical of AD. Cholinesterase inhibitors inhibit the degradation of acetylcholine released into the synaptic clefts of the brain, maintaining acetylcholine concentration within the brain and thereby enhancing cholinergic neurotransmission. As an example, degradation of extracellular acetylcholine is typically by acetyl- and/or butyrylcholinesterase. Rivastigmine (Exelon) is a cholinesterase inhibitor that is FDA-approved to treat all stages of AD.

Thus, exemplary drugs for nose-to-brain drug delivery, include Rivastigmine. Rivastigmine administered through nasal tissue in a delivery device of the present inventions for continuous delivery over time, has the potential to address these limitations and improve efficacy and safety by providing continuous therapeutic dosing at a steady rate without the need for patient compliance, see below for additional benefits. Merely as exemplary examples, olfactory cleft placement of a device configured to target drug elution into olfactory nerve pathways; into middle meatus (MM) placement to target drug elution into trigeminal and olfactory nerve pathways.

Rivastigmine is used to treat dementia (a brain disorder that has symptoms of and affects the ability to remember, think clearly, communicate, and perform daily activities and may cause changes in mood and personality) in people with Alzheimer's disease (a brain disease that slowly destroys the memory and ability to think, learn, communicate and handle daily activities).

Rivastigmine is used to treat Lewy body dementia (a condition in which the brain develops abnormal protein structures, and the brain and nervous system are destroyed over time). Rivastigmine is used to treat dementia in people with Parkinson's disease (a brain and nervous system disease with symptoms of slowing of movement, muscle weakness, shuffling walk, and loss of memory). It improves mental function (such as memory and thinking) by increasing the amount of a certain natural substance in the brain.

Rivastigmine, as a dual cholinesterase inhibitor, is approved for all stages of AD and is available both as oral and transdermal patch formulations. Oral and transdermal patch doses of Rivastigmine are limited by systemic tolerability, such that maximal therapeutic levels are not achieved in the brain using either of these routes of administration. The oral formulation, for one example, is associated with a high incidence of gastrointestinal problems among patients, so that dosage must be increased slowly over several weeks to achieve a therapeutic effect while attempting to promote tolerance and prevent severe adverse events.

Further, oral administration may have first-pass metabolism effects. This lag time results in a significant lag between treatment onset and achieving a therapeutic concentration of the drug in the brain. A transdermal patch formulation is better tolerated, though adverse events related to the systemic route of administration are still observed, e.g., first-pass metabolism effects. Regardless of formulation, the therapeutic effect of rivastigmine may be compromised by poor patient compliance to the recommended daily dosing regimens. Cognitive impairments of AD and other types of CNS disorders, diseases and injuries, may render patient adherence to treatment a significant problem. Moreover, rivastigmine overdose can be fatal, e.g., from improper patch administration, further highlighting the importance of proper administration of rivastigmine. Further, monitoring rivastigmine administration, especially in a patient population suffering from dementia, poses a significant burden for caretakers and patients.

Thus, rivastigmine's peripheral side effects, augmented by issues with safety/tolerability and compliance, may limit the ability to achieve optimal therapeutic benefit in patients with AD with current administrative routes, not using devices as described herein. Thus, there is a significant unmet medical need for a route of administration of rivastigmine that provides long-term, continuous delivery of therapeutic doses of a drug to the brain with limited systemic exposure. More specifically, there is an unmet medical need for a delivery device that provides long-term, continuous delivery of therapeutic doses of rivastigmine to the brain with limited systemic exposure to improve patient outcomes.

To address this unmet medical need for continuous delivery of therapeutic agents to CNS target tissues, e.g., nose-to-brain delivery, a CNS delivery device is described herein.

A CNS delivery coated matrix (mesh) scaffold device is configured to self-expand after implantation, to conform to the target nasal anatomy and maintains proper positioning over the treatment period. Proper positioning provides persistent positioning over the treatment period. To deliver rivastigmine directly to the central nervous system (CNS), a coated matrix scaffold device eluting rivastigmine will be configured to fit within the nasal cavity, e.g. within an olfactory cleft, within a MM, etc., to enhance delivery of rivastigmine to the brain. As one example, such nose to brain delivery will allow an active agent to access the brain through olfactory and/or trigeminal nerve pathways, etc., while minimizing systemic absorption. This route of administration is contemplated to avoid first-pass metabolism and is contemplated to bypasses the blood-brain barrier, which may limit systemic exposure and reduce the dosage required for a therapeutic effect. Therefore, delivery of drugs on a modified CNS delivery device/device for a continuous release of rivastigmine over time, has the potential to improve the standard of care and improve patient outcomes by reducing the incidence of rivastigmine-associated adverse events, improving patient compliance, and accelerating the accumulation of a therapeutic concentration of rivastigmine in the brain. More specifically, such a CNS delivery device has the potential to dramatically improve the standard of care for AD by reducing the incidence of rivastigmine-associated adverse events and accelerating the accumulation of a therapeutic concentration of rivastigmine in the brain while improving patient compliance. Compositions and methods contemplate applying the CNS delivery device technology to provide nose-to-brain delivery of an active agent, such as a drug, e.g., rivastigmine as described herein.

Additional active ingredients that may be included, either administered alone before, or after device is removed or during the time period the device is present in a nasal tissue, or administered using a CNS delivery device as described herein, including, but are not limited to, anticholinergic agents, antihistamines, anti-infective agents, anti-inflammatory agents, anti-scarring or antiproliferative agents, chemotherapeutic/antineoplastic agents, cytokines such as interferon and interleukins, decongestants, healing promotion agents and vitamins (e.g., retinoic acid, vitamin A, and their derivatives), hyperosmolar agents, immunomodulator/immunosuppressive agents, leukotriene modifiers, mucolytics, narcotic analgesics, small molecules, tyrosine kinase inhibitors, peptides, proteins, nucleic acids, vasoconstrictors, or combinations thereof. Anti-sense nucleic acid oligomers or other direct transactivation and/or transrepression modifiers of mRNA expression, transcription, and protein production may also be used. Anti-infective agents generally include antibacterial agents, antifungal agents, antiparasitic agents, antiviral agents, and antiseptics. Anti-inflammatory agents generally include steroidal, nonsteroidal anti-inflammatory agents and monoclonal antibodies, etc.

Examples of antibacterial agents that may be suitable for use with a CNS delivery device as described herein, include, but are not limited to, aminoglycosides, amphenicols, ansamycins, β-lactams (such as carbacephems, carbapenems, cephalosporins, cephamycins, monobactams, oxacephems, penicillins, and any of their derivatives), lincosamides, macrolides, nitrofurans, quinolones, sulfonamides, sulfones, tetracyclines, vancomycin, and any of their derivatives, or combinations thereof.

Examples of antifungal agents suitable for use with a CNS delivery device as described herein, include, but are not limited to, allylamines, imidazoles, polyenes, thiocarbamates, triazoles, and any of their derivatives. Antiparasitic agents that may be employed include such agents as atovaquone, clindamycin, dapsone, iodoquinol, metronidazole, pentamidine, primaquine, pyrimethamine, sulfadiazine, trimethoprim/sulfamethoxazole, trimetrexate, and combinations thereof.

Examples of antiviral agents suitable for use with a CNS delivery device as described herein, include, but are not limited to, acyclovir, famciclovir, valacyclovir, edoxudine, ganciclovir, foscamet, cidovir (vistide), vitrasert, formivirsen, HPMPA (9-(3-hydroxy-2-phosphonomethoxypropyl)adenine), PMEA (9-(2-phosphonomethoxyethyl)adenine), HPMPG (9-(3-Hydroxy-2-(Phosphonomet-hoxy)propyl)guanine), PMEG (9-[2-(phosphonomethoxy)ethyl]guanine), HPMPC (1-(2-phosphonomethoxy-3-hydroxypropyl)-cytosine), ribavirin, EICAR (5-ethynyl-1-beta-D-ribofuranosylimidazole-4-carboxamine), pyrazofurin (3-[beta-D-ribofuranosyl]-4-hydroxypyrazole-5-carboxamine), 3-Deazaguanine, GR-92938X (1-beta-D-ribofuranosylpyrazole-3,4-dicarboxami-de), LY253963 (1,3,4-thiadiazol-2-yl-cyanamide), RD3-0028 (1,4-dihydro-2,3-Benzodithiin), CL387626 (4,4′-bis[4,6-d][3-aminophenyl-N—, N-bis(2-carbamoylethyl)-sulfonilimino]-1,3,5-triazin-2-ylamino-biphenyl-2-,2′-disulfonic acid disodium salt), BABIM (Bis[5-Amidino-2-benzimidazoly-1]-methane), NIH351, and combinations thereof.

Examples of steroidal anti-inflammatory agents that may be used with a CNS delivery device as described herein, include 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, any of their derivatives, and combinations thereof. In one variation, the steroidal anti-inflammatory agent may be mometasone furoate. In another variation, fluticasone propionate may be included in the systems as the steroidal anti-inflammatory agent.

Suitable nonsteroidal anti-inflammatory agents include, but are not limited to, COX inhibitors (COX-1 or COX nonspecific inhibitors) (e.g., salicylic acid derivatives, aspirin, sodium salicylate, choline magnesium trisalicylate, salsalate, diflunisal, sulfasalazine and olsalazine; para-aminophenol derivatives such as acetaminophen; indole and indene acetic acids such as indomethacin and sulindac; heteroaryl acetic acids such as tolmetin, dicofenac and ketorolac; arylpropionic acids such as ibuprofen, naproxen, flurbiprofen, ketoprofen, fenoprofen and oxaprozin; anthranilic acids (fenamates) such as mefenamic acid and meloxicam; enolic acids such as the oxicams (piroxicam, meloxicam) and alkanones such as nabumetone) and selective COX-2 inhibitors (e.g., diaryl-substituted furanones such as rofecoxib; diaryl-substituted pyrazoles such as celecoxib; indole acetic acids such as etodolac and sulfonanilides such as nimesulide).

Chemotherapeutic/antineoplastic agents that may be used with a CNS delivery device as described herein, include, but are not limited to antitumor agents (e.g., cancer chemotherapeutic agents, biological response modifiers, vascularization inhibitors, hormone receptor blockers, cryotherapeutic agents or other agents that destroy or inhibit neoplasia or tumorigenesis) such as alkylating agents or other agents which directly kill cancer cells by attacking their DNA (e.g., cyclophosphamide, isophosphamide), nitrosoureas or other agents which kill cancer cells by inhibiting changes necessary for cellular DNA repair (e.g., carmustine (BCNU) and lomustine (CCNU)), antimetabolites and other agents that block cancer cell growth by interfering with certain cell functions, usually DNA synthesis (e.g., 6 mercaptopurine and 5-fluorouracil (5FU), antitumor antibiotics and other compounds that act by binding or intercalating DNA and preventing RNA synthesis (e.g., doxorubicin, daunorubicin, epirubicin, idarubicin, mitomycin-C and bleomycin) plant (vinca) alkaloids and other anti-tumor agents derived from plants (e.g., vincristine and vinblastine), steroid hormones, hormone inhibitors, hormone receptor antagonists and other agents which affect the growth of hormone-responsive cancers (e.g., tamoxifen, herceptin, aromatase ingibitors such as aminoglutethamide and formestane, triazole inhibitors such as letrozole and anastrazole, steroidal inhibitors such as exemestane), antiangiogenic proteins, small molecules, gene therapies and/or other agents that inhibit angiogenesis or vascularization of tumors (e.g., meth-1, meth-2, thalidomide), bevacizumab (Avastin), squalamine, endostatin, angiostatin, Angiozyme, AE-941 (Neovastat), CC-5013 (Revimid), medi-522 (Vitaxin), 2-methoxyestradiol (2ME2, Panzem), carboxyamidotriazole (CAI), combretastatin A4 prodrug (CA4P), SU6668, SU11248, BMS-275291, COL-3, EMD 121974, IMC-1C11, IM862, TNP-470, celecoxib (Celebrex), rofecoxib (Vioxx), interferon alpha, interleukin-12 (IL-12) or any of the compounds identified in Science Vol. 289, Pages 1197-1201 (Aug. 17, 2000), which is expressly incorporated herein by reference, biological response modifiers (e.g., interferon, bacillus calmette-guerin (BCG), monoclonal antibodies, interluken 2, granulocyte colony stimulating factor (GCSF), etc.), PGDF receptor antagonists, herceptin, asparaginase, busulphan, carboplatin, cisplatin, carmustine, chlorambucil, cytarabine, dacarbazine, etoposide, flucarbazine, fluorouracil, gemcitabine, hydroxyurea, ifosphamide, irinotecan, lomustine, melphalan, mercaptopurine, methotrexate, thioguanine, thiotepa, tomudex, topotecan, treosulfan, vinblastine, vincristine, mitoazitrone, oxaliplatin, procarbazine, streptocin, taxol or paclitaxel, taxotere, analogs/congeners, derivatives of such compounds, and combinations thereof.

### Exemplary Devices

Although it is not intended to limit the time over which drugs may elute from a device described herein, in one preferred embodiment there is a continuous release of rivastigmine up to 30 days or more, up to 12 weeks, 24 weeks, 30 weeks, in vitro.

One exemplary embodiment of the present implantable devices is a device comprising of one or more fibers, at least one of which is a permeable, hollow fiber comprising an agent or active ingredient. This device, or scaffold, is not limited to the number of fibers or structure the fibers take. Another exemplary embodiment of the present implantable devices is a device comprising a permeable or semi-permeable, sheet, which contains an active ingredient. The fiber or sheet may be considered a permeable or semi-permeable membrane.

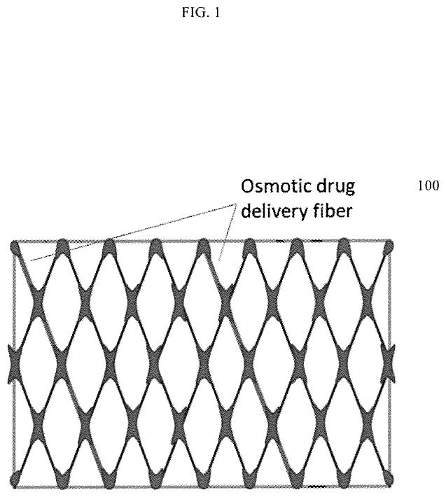

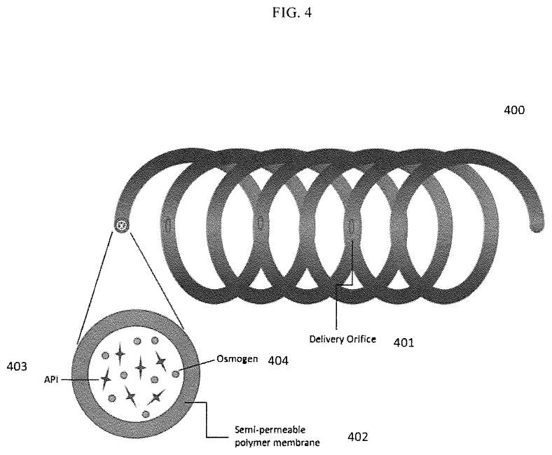

The embodiment of the device comprising one or more fibers contains osmotic drug delivery components. In this exemplary embodiment, drug delivery components are comprised of one or more permeable or semi-permeable polymeric, hollow fibers filled with a drug or active pharmaceutical ingredient (API) in the absence or presence of osmogens. The present invention is not limited by the number or arrangement of the fiber(s). In one embodiment, fiber arrangement is a spiral, as seen in FIG. 4. One embodiment, the fiber arrangement is braided. In this design, shown in FIG. 1, the implants comprise a fiber-based braid structure with multiple strands (e.g. 2 to 64), where at least one fiber comprises semi-permeable membrane that encapsulates the API(s).

The embodiment of the device comprising a sheet may contain osmotic drug delivery components. The permeable or semi-permeable sheets may be implanted flat or in a rolled state. In the rolled embodiment, the rolled sheet comprises an internal lumen. In this exemplary embodiment, drug delivery components are comprised of a semi-permeable polymeric hollow sheet filled with a drug or active pharmaceutical ingredient (API) in the absence or presence of an osmogen.

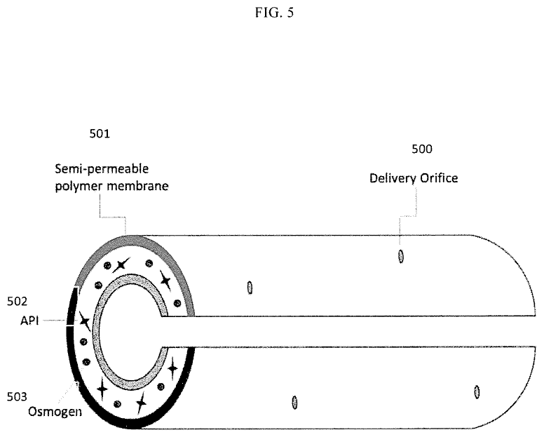

The implantable device may comprise a permeable or semi-permeable membrane, such as one or more fibers or a sheet, as seen in FIG. 5. In one embodiment, permeability to fluid is achieved through the use of permeable materials. In another embodiment, permeability is achieved through one or more delivery orifices on the hollow fiber or sheet wall. Any number of orifices is contemplated, including, but not limited to, one, two, three, four, five, six, seven, eight, nine, ten, twenty-five, fifty, one hundred, two hundred, a thousand, etc.

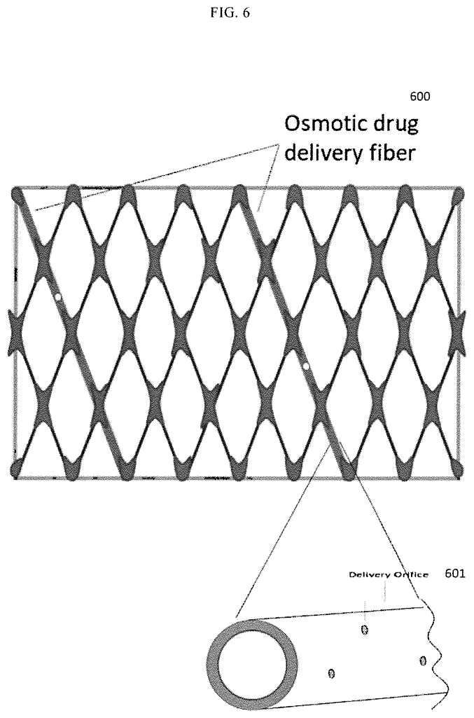

In one embodiment, the devices herein may be coated or covered. It is not intended for the present invention to be limited by the type, thickness, or coverage of the coating, such as an elastomer. The device may be completely or partially coated. In one embodiment, there may be an elastomer coating on the top of the permeable or semi-permeable membrane, such as the hollow fibers or sheet, covering or not covering any delivery orifices, as seen in FIG. 6. Elastomers may be coated onto the implants to provide them with self-expandability. One or more orifices may be formed on the semi-permeable membrane either before or after the elastomer coating.

In one embodiment, the device may be expandable. In one embodiment, the device may be self-expanding. In one embodiment, the device may be balloon-expandable. The many scaffold embodiments of the present disclosure may be self-expanding in that they are manufactured at a first diameter, subsequently reduced or “crimped” to a second, reduced diameter for placement within a delivery catheter, and self-expand towards the first diameter when extruded from the delivery catheter at an implantation site. The first diameter may be at least 10% larger than the diameter of the bodily lumen into which it is implanted in some embodiments. The scaffold may be designed to recover at least about 70%, at least about 80%, at least about 90%, up to about 100% of its manufactured, first diameter, in some embodiments.

In one embodiment, the device may be biodegradable or biodurable or bioabsorbable.

In one embodiment, various components of the device may be hydrophilic, hydrophobic, lipophilic, etc.

Upon implantation, a fluid, such as water, enters the lumen through the permeable or semi-permeable wall, forming an osmotic pressure gradient that pushes the active pharmaceutical ingredient (API) out of the delivery orifices at a steady rate. These osmotic dosage forms function by allowing a fluid, such as water, around the implant to flow through the semi-permeable membrane, dissolve the API in the core so it can be released through the ports in the membrane by the osmotic pressure.

The present devices and systems may be used with a large multitude of active ingredients. Agents, such as active pharmaceutical ingredients (APIs), may be embedded in porous or semi-porous fiber strands or sandwiched in porous or semi-porous sheets. In one embodiment, the agent is an active pharmaceutical ingredient. In one embodiment the present agent is a therapeutic agent. In one embodiment, the present agent is a glucocorticoid. In one embodiment, the present agent is a drug.

The polymers used in the implants can be biodegradable, nonbiodegradable or biodurable. Polymers used in the implantable device include cellulose esters, alkyl-celluloses, and cellulose derivatives including methylcellulose, ethyl cellulose, hydroxyethyl cellulose, hydroxypropyl cellulose, hydroxylpropyl methyl cellulose, cellulose nitrate, cellulose acetate ethyl carbamate, cellulose acetate phthalate, cellulose acetate dimethaminoacetate, cellulose acetate ethyl carbonate, cellulose acetate chloroacetate, cellulose acetate ethyl oxalate, or any combination of any thereof. Synthetic polymers that may be used in the present device include partially and completely hydrolyzed alkylene-vinyl acetate copolymers, hydroxylated and unhydroxylated ethylene-vinyl acetate copolymers, derivatives of polystyrene such as poly(sodium styrenesulfonate) and poly(vinylbenzyltrimethylammonium chloride), homo- and copolymers of polyvinyl acetate, polymers of acrylic acid and methacrylic acid, copolymers of an alkylene oxide and alkyl glycidyl ether, polyurethanes, polyamide, polyshulphones, crosslinked poly(ethylene oxide), poly(alkylenes), poly(vinyl imidazole). Semi-permeable bioresorbable polymers that may be used in the present device include polyglycolic acid, polylactic acid, polycaprolactone, polydioxanone, poly(trimethylene carbonate), poly(3-hydroxybutyrate), poly(propiolactone), poly(ethylene succinate), poly(butylenes succinate), poly(3-hydroxybutyrate-co-3-hydroxyvalerate), poly(ester carbonate), poly(glycerol sebacate), and their copolymers and derivatives thereof.

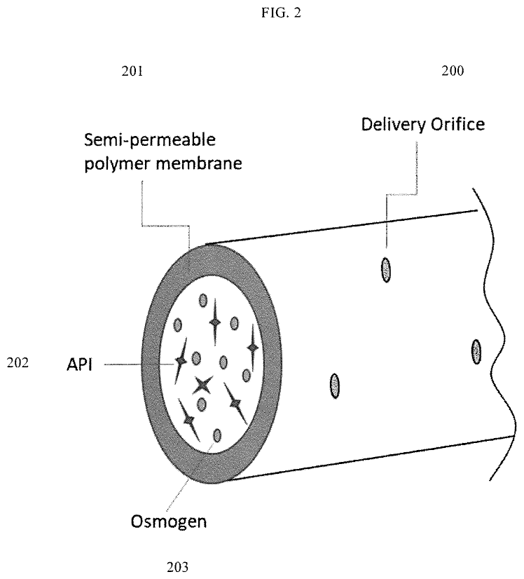

The device may comprise a variety of substances, as seen in FIG. 2, such as the API, osmogens, and other aiding agents (e.g. wicking agents, surfactants, and solubilizing agents), while the shell is comprised of the semi-permeable polymer. Various osmogens, or osmotic agents, can be used to tailor the osmotic pressure inside the semi-permeable membrane and consequently the drug release rate. These osmogens include but not limited to sodium chloride, potassium chloride, potassium sulfate, sodium phosphate, fructose, sucrose, glucose, lactose, dextrose, xylitol, sorbitol, mannitol, citric acid, tartaric acid, fumaric acid, adipic acid, and their combinations at any ratios. The osmogen can also be a water-soluble organic polymer such as hydroxy propyl methyl cellulose (HPMC), sodium carboxy methyl cellulose (Na CMC), polyethylene oxide (PEO), polyvinyl pyrrolidine (PVP), and methyl cellulose (MC) or a water-soluble amino acid such as alanine, glycine, leucine, and methionine.

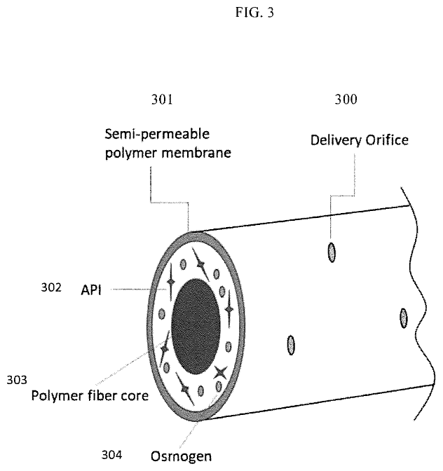

The present invention is advantageous as it can be formed through a variety of manufacturing methods, such as coextrusion, filling, or successive coating. In one embodiment, drug-encapsulated fibers are formed by coextrusion of the API(s) and the semi-permeable polymers into a core-shell structure of extrusion. In another embodiment, drug-encapsulated fibers are formed by first hollow fibers comprising a lumen, followed by filling the lumen with API(s). As seen in FIG. 2, the lumen of the osmotic drug delivery fiber may comprise a variety of substances, such as the API, osmogens, and other aiding agents (e.g. wicking agents, surfactants, and solubilizing agents), while the shell may be comprised of the semi-permeable polymer. In another embodiment, the drug-encapsulated fibers are formed by coating a solid polymer fiber core successively with the API(s) and a semi-permeable polymer membrane. As shown in FIG. 3, the APIs are encapsulated in between the polymer fiber core and the semi-permeable polymer membrane to form a sandwich structure. The two ends of the fiber comprising API may be blocked through polymer coating or welding. Following the fabrication of the individual fibers, one or more fibers, some containing API and some not, can be formed into an implant or scaffold.

One or more fibers described above can be fabricated into spiral scaffolds, with at least one comprising API, as seen in FIG. 4. Following the blockage of the ends of the fibers, one or more drug delivery orifices may be placed on the semi-permeable wall using, for example, laser drilling. A shape memory polymer fiber may be attached to the side or even serve as the core of the drug delivery fiber, to maintain the spiral shape of the fiber after implantation. In addition, an elastomer can be coated onto the spiral scaffolds to enhance their recoverability post implantation.

Multi-stranded scaffolds comprising other fiber arrangements are manufactured following fabrication of single fibers, comprising at least one strand of those API-encapsulated fibers. The fibers may be arranged in a spiral, as stated above, or a braid, mesh, etc. The scaffolds can be conformally coated with an elastomer to provide the scaffold self-expandability. Following the blockage of the ends of the fibers, one or more drug delivery orifices may be placed on the semi-permeable wall using, for example, laser drilling.

Before or after such elastomer coating or arrangement of fibers, one or more delivery orifices are introduced onto the semi-permeable membrane of each API-encapsulated fiber through either mechanical drilling or laser drilling. Either luminal or abluminal delivery orifices can be formed accordingly. The size, density, and location of the delivery orifices are determined by the API used, the target implantation sites, and the dosing requirement. Furthermore, the delivery orifices can also be formed by a salt-leaching approach, where inorganic salt granules are present during the semi-permeable membrane formation. Upon implantation, the salt will dissolve and leach out to form drug delivery orifices in situ. The number and size of the orifices can be tuned by tailoring the size and quantity of salt granules within the membrane.

The sheet embodiment also may be manufactured in a variety of methods. APIs and aiding agents are encapsulated in between two polymer membranes to form a drug release sheet. One or both polymer membranes are semi-permeable membranes. Drug delivery orifices can be drilled on either polymer membranes to allow drug release. Optional elastomer coating can be further introduced onto the rolled sheets to improve their self-expandability.

Drug coating and formulation development. The absolute bioavailability of oral rivastigmine is approximately 40%, and the AUC_{1-12 h} cerebral spinal fluid (CSF)/plasma ratio is about 40% (Novartis Exelon® Capsules and Oral Solution Prescribing Information). An estimated 0.87% of the rivastigmine oral dose is absorbed and distributed into the CSF. Thus, the daily CSF uptake of this drug is estimated to be between 26-105 μg. As a starting point for the formulation development, we will first target to formulate a matrix loaded with rivastigmine which will be released over 30 days in a near linear manner. Bilateral administration of the rivastigmine matrix would result in approximately 21-107 μg rivastigmine delivered to CSF/day, assuming a CSF bioavailability between 10-50%.

Rivastigmine will be formulated onto the base structure using carrier polymers such as poly(L-lactide), poly(glycolide), poly(ε-caprolactone), and their copolymers and blends. While rivastigmine is commonly supplied as a tartrate salt, the salt form is highly water-soluble, complicating long-term release. The nonionized form of rivastigmine (free base) will be used instead. We will incorporate rivastigmine into a polymeric coating on the top of the base structure by a spray- or dip-coating process. A drug-free topcoat may be applied on the top of the drug layer to further control the drug release by diffusion-dominated kinetics. The near linear release profile of rivastigmine over ˜30 days will be achieved by tuning the following parameters: (1) rivastigmine to carrier polymer ratio and drug coating thickness; (2) topcoat material composition and thickness; (3) incorporation of biocompatible polyanionic polymers such as poly(methylmethacrylate-co-methacrylic acid) and poly(carboxyalkyl methacrylates) as a drug carrier; and (4) incorporation of inactive ingredients like lauric acid (C12), stearic acid (C18), and 9-hexadecenoic acid to control the diffusion rate of rivastigmine. Previous studies indicate that adjusting parameters (1) and (2) alter the release profiles of paclitaxel or MF. Further, incorporation of hydrophobic and acidic inactive ingredients provides another lever to control the release rate of the inherently basic rivastigmine. Inactive ingredients such as tocopherol (Vitamin E), butylhydroxytoluene, and propyl gallate may also be formulated into the system to enhance rivastigmine stability.

### EXAMPLES

The following examples are provided for supporting the use of nose-to-brain CNS drug delivery as described herein, for treating an exemplary disease, e.g., Alzheimer's Disease. A modified XTREO™ technology platform is contemplated for delivery of rivastigmine.

### Example 1

### Rivastigmine for Treating Alzheimer's Disease.

Nose-to-brain drug delivery, e.g., using a CNS agent delivery device as described herein, addresses a significant unmet medical need for continuous and long-term treatment of CNS diseases and disorders, such as Alzheimer's Disease. As one example, there is a need to develop a long-acting, implantable nose-to-brain drug delivery platform for continuous delivery of rivastigmine for treating Alzheimer's Disease (AD).

Delivery and biodistribution of intranasally-administered active agents will be measured in treated patients in vivo, e.g., Rivastigmine, AVP-786, deuterated dextromethorphan (without the quinidine), and other drugs.

Formulations of rivastigmine for nose to brain delivery, e.g., as a CNS agent delivery mesh scaffold device will be developed, wherein the delivery mesh device comprises rivastigmine (or related formulation), to provide potentially beneficial continuous drug delivery for treating a CNS disease, such as AD.

As one step towards applying a CNS delivery device to the delivery of active agents, including drugs, for relieving or slowing AD progress, via the nasal cavity, formulations of rivastigmine will be tested for elution rates after implantation. In one contemplated embodiment, a biocompatible mesh scaffold comprising a rivastigmine formulation that elutes rivastigmine at a near linear rate up to 24 days, and up to or over 30 days in vitro.

In one embodiment, a CNS drug delivery device, is a matrix (implant) comprising braided monofilament polymer fibers coated with elastomer configured for allowing continuous delivery of a drug formulation. One nonlimiting example of a drug formulation comprises 0.8 mg-3.2 mg of rivastigmine per implantable matrix designed to release drug over 30 days. Thus, in one embodiment, there is contemplated a continuous release of rivastigmine up to and over 30 days in vitro.

### Exemplary Rivastigmine Formulations

An oral dose of rivastigmine for treating a disease, e.g., AD, typically starts around 1.5 mg BID (i.e., b.i.d. or “bis in die” refers to twice (two times) a day) for 2 weeks and then gradually increases to higher doses (3 mg BID, 4.5 mg BID, and 6 mg BID) every 2 weeks.

For oral administration, delivery, an absolute bioavailability of Rivastigmine is about 40% and a mean AUC (area under the curve) 1-12 hr CSF/Plasma is about 40% (Exelon Label). The daily doses delivered to CSF are 26 μg/day, 52 μg/day, 79 μg/day, and 105 μg/day for the oral doses, equating to 1.5 mg BID, 3 mg BID, 4.5 mg BID, and 6 mg BID, respectively. For this estimation, a plasma volume of 2750 mL and a CSF volume of 150 mL are used.

Estimating at least 50% of a drug is delivered to CSF through (eluted from) a coated matrix, a formulation is then estimated as a daily dose of 26 μg/day per coated matrix to 105 μg/day per matrix. Thus, for preparing an exemplary delivery matrix of up to a 30-day or more drug delivery, the drug load per matrix will be approximately between, preferably 0.1 mg/matrix, but more preferably 0.8 mg/matrix and 3.2 mg/matrix (e.g., as targets for initial formulation development). Drug load may be increased or decreased, depending upon one or more of measurements of actual delivery to target tissue(s); patient response. For examples, in some embodiments, for drug release up to 4 weeks, up to 8 weeks, up to 12 weeks, up to 16 weeks, up to 20 weeks, up to 24 weeks, up to 30 weeks or more, amounts of drugs loaded onto a CNS delivery device may be adjusted for linear release over these longer time periods.

### Example 2

### Evaluate Rivastigmine Delivery to the Brain In Vivo Via a CNS Delivery Device as Described Herein.

Developing a mammalian model is contemplated, e.g., rabbit, rodent (e.g., mouse, rat) model, to assess nose-to-brain drug delivery with a modified XTREO™ platform. In one embodiment, a rabbit model will be developed, deployed and analyzed after implantation of a CNS drug delivery device into a nasal cavity. In another embodiment, a mammalian model, e.g., rabbit or rat model will be used. In another embodiment, analysis of a CNS drug delivery device will be evaluated after implantation into a human patient.

A. Develop a Formulation of Rivastigmine on the CNS Delivery Device for Continuous Drug Delivery.

In order to determine delivery and biodistribution of intranasally-administered rivastigmine in vivo, a mammalian model for a human patient, e.g., rabbit, rat, etc., may be used or testing implantation of a modified CNS delivery device into a nasal cavity. This will be done by investigating the optimal placement locations, e.g. olfactory cleft, mm, etc., and the appropriate product dimensions of the implantable device, and corresponding implantation device for the delivery device, in mammals, such as humans, rabbits, rats etc. In one embodiment, said device will expand to be located adjacent to and in contact with tissues for elution of drug directly into nasal tissues. In one embodiment, said device will expand to be located adjacent to and in contact with placement in the maxillary sinus tissues.

Each implant comprises at least one coating, said coating containing a drug and/or agent. In one preferred embodiment, said coating is a polymer coating. In a further embodiment, the drug containing coating is overlaid (at least in part) with another polymer coating or “topcoat” lacking drug. In one embodiment, the thickness of the topcoat controls the amount and/or timing of drug release. Moreover, a CNS delivery device comprises a drug containing coating is overlaid (at least in part) with another polymer coating or “topcoat” lacking drug. In one embodiment, the thickness of the topcoat controls the amount and/or timing of drug release. In one embodiment, a CNS delivery device comprising rivastigmine is overlain with a topcoat for eluting rivastigmine at a near linear rate over 30 days in vitro.

For the placement of an implantable mesh (matrix) implant into a particular nasal cavity, e.g., olfactory cleft, MM, etc., an in vivo study will be used to assess nose-to-brain drug delivery over time in a mammal, such as a rabbit, rodent model and in human patients.

As part of evaluation of devices, delivery of drug to target tissues, amounts of drugs, and/or metabolic by products, within blood samples, will be compared between devices implanted in different parts of nasal openings, e.g., olfactory cleft vs. mm. In some embodiments, said device will implanted into the maxillary sinus. In some embodiments, when desired, different types of coatings may be compared for drug release.

Measurements will be taken for determining the concentration of rivastigmine in the brain and rivastigmine in the plasma over time. The patients will be monitored for any signs of systemic toxicity. Measurements and/or evaluation will include, but not limited to: dose-ranging comparisons, safety to tissue adjacent to implant (e.g., olfactory epithelium), biodistribution in the brain, plasma kinetics (PK), head-to-head (direct comparisons to patients or models) treated with the same systemic drug. In one embodiment, blood samples, and/or fluid and/or tissue samples will be collected, before, during and after treatments. Thus, samples will be taken for maturing amounts of compounds as described herein.

In some embodiments, measurements and/or evaluations of patients treated with a CNS delivery device will be compared to known therapeutic dose/concentration in the brain and/or bodily fluids, e.g., CNSF, blood etc., of patients treated with oral, patch, i.v., etc administration.

Furthermore, a CNS-specific and systemic delivery of rivastigmine via modified XTREO™ platforms, including modifications of implantable devices to allow described delivery, e.g., desired release kinetics, in vivo.

In one embodiment, a CNS delivery modified XTREO™ platform will be implanted into a mammal, e.g., human, rabbits, rodents, e.g., mice, rats, using methods described herein.

In one embodiment, a rivastigmine formulation containing delivery device will be implanted in the nasal cavity for delivery of rivastigmine to the brain.

In one embodiment, an in vivo assessment and characterization of rivastigmine delivery over longer time periods, up to 30 weeks or more, are contemplated using a delivery device as described herein. Additional measurements and evaluations include but not limited to, local and systemic safety, pharmacokinetics, drug distribution in in the brain, acetylcholine levels in the brain (an indicator of functional blockade of rivastigmine), and comparison of intranasal vs. oral administration of rivastigmine delivery in rodents.

B. Assess In Vivo CNS and Systemic Delivery of Rivastigmine Via a CNS Delivery Device Compared to Standard Administration of Rivastigmine.

An implantable CNS agent delivery mesh device having a coating comprising a rivastigmine formulation, after it alone or as two implants, configured for a rabbit (or other mammal) nasal cavity is implanted into the nasal cavity for testing. It will be determined whether it delivers rivastigmine to the brain and alters the central nervous system (CNS) and systemic pharmacokinetic profiles compared to after oral rivastigmine administration. For exemplary examples, rivastigmine concentration in the brain and plasma over 48 hours measured (e.g. LC-MS/MS of brain fluid, brain tissue, and plasma samples). Rivastigmine concentrations are contemplated to increase in the brain and decreased in the plasma when delivered (administered) via a CNS delivery device compared to oral administration of rivastigmine over up to 24, and up to 48 hours or more. As one exemplary example, detection of persistent rivastigmine concentrations in the brain will be measured after 48 hours

To assess brain delivery of rivastigmine using the XTreo matrix, we will use an established New Zealand White Rabbit (NZWR) model. Rivastigmine will be administered to a group of NZWRs (n=8) via XTreo matrix implanted in the nasal cavity.²⁹ Briefly, NZWRs are anesthetized, and surgical access to the maxillary sinus is achieved via bilateral burr hole osteotomy to implant the XTreo-rivastigmine matrix. Endoscopy (pre- and post-implant) will be used to assess matrix placement. Following recovery from anesthesia, NZWRs will receive a veterinary clinical assessment, including neurological assessments. A second group of NZWRs (n=8) will receive two oral doses of rivastigmine (0 and 24 hours). Blood samples will be collected at multiple time points (baseline, 6 hr, 24 hr, 30 hr and 4810 to measure plasma rivastigmine levels. NZWRs (n=4/group, 8 total) will be euthanized at 24 and 48 hours for brain tissue collection and bioanalytical measurement of rivastigmine drug levels. Pre-termination nasal endoscopy will be performed to assess matrix retention. We have demonstrated that XTreo matrix placement in the NZWR nasal cavity is well tolerated.²⁹ To assess the tolerability of XTreo-rivastigmine, we will employ clinical observation, serial nasal endoscopy, and treatment site observation at necropsy.

Data analysis plan. Rivastigmine concentrations over time and rivastigmine concentrations in the brain versus in plasma will be the key data for analysis and interpretation. PK data will be analyzed utilizing standard data analysis software (e.g., WIN-NONLIN).

### Example 3

### Treating CNS Diseases and Disorders

AVP-786 refers to a drug formulation currently used for treatment of patients having AD, dementias, and the like, including agitation in patients with dementia of the Alzheimer's type, in addition to many other types of brain or SP (spinal cord) injuries and CNS diseases/disorders. AVP-786 formulations are used for oral administration for treating agitation, schizophrenia, brain injuries, impulse control disorders, major depressive disorders, neurodegenerative disorders, etc.

Thus, in some embodiments, an AVP-786 formulation, and parts of this formulation, e.g., dextromethorphan (DXM) or deuterated dextromethorphan (in both cases, without the quinidine) are contemplated for use in a CNS delivery device as described herein.

Exemplary oral capsules include 20 mg d-DXM and 10 mg Q or 30 d-DXM mg 10 mg Quinidine once daily.

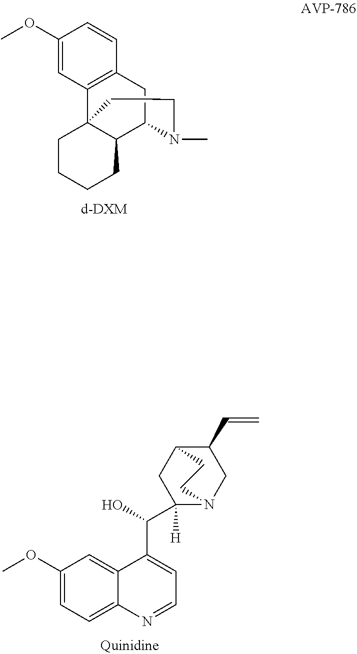

AVP-786 is administered as a combination pharmaceutical compound product because quinidine inhibits the rapid first-pass metabolism of deuterated-dextromethorphan (d-DXM) into its inactive form. In one contemplated embodiment for nose-to-brain delivery, is delivery of the dextromethorphan or deuterated dextromethorphan (without the quinidine), since this should be bypassing a first-pass metabolism by the BBB. The d-DXM molecule shown above is compatible with a modified XTREO™ platform.

NEUROPSYCH: Deudextromethorphan/quinidine (d-DXM/Q; developmental code names AVP-786, CTP-786) is a combination of deudextromethorphan (d-DXM; deuterated (d6) dextromethorphan (DXM)) and quinidine (Q) which is under development by Avanir Pharmaceuticals for the treatment of a variety of neurological and psychiatric indications. The pharmacological profile of d-DXM/Q is similar to that of dextromethorphan/quinidine (DXM/Q). DXM and d-DXM act as σ1 receptor agonists, NMDA receptor antagonists, and serotonin-norepinephrine reuptake inhibitors, among other actions, while quinidine is an antiarrhythmic agent acting as a CYP2D6 inhibitor. Quinidine inhibits the metabolism of DXM and d-DXM into dextrorphan (DXO), which has a different pharmacological profile from DXM. Deuteration of DXM hinders its metabolism by CYP2D6 into DXO, thereby allowing for lower doses of quinidine in the combination. This in turn allows for a lower potential for drug interactions and cardiac adverse effects caused by quinidine. Thus, another beneficial effect of to the patient by using a CNS drug delivery device, as described herein, by avoiding the use of or using a lower amount of quinidine.

Although various embodiments are specifically illustrated and described herein, it will be appreciated that modifications and variations of the present disclosure are covered by the above teachings and are within the purview of the appended claims without departing from the spirit and intended scope of the disclosure.

1. A method of treating a central nervous system condition, comprising:
a) providing an implant comprising a therapeutic compound; and
b) implanting said implant at a position inside the nose of a patient having a symptom of a central nervous system condition, wherein said position allows for delivery of the therapeutic compound to the brain.

2. The method of claim 1, wherein said implant comprises a polymer scaffold.

3. The method of claim 2, wherein said polymer scaffold comprises fibers.

4. The method of claim 1, wherein said implant releases said therapeutic compound by osmosis.

5. The method of claim 1, wherein said therapeutic compound is a cholinesterase inhibitor.

6. The method of claim 5, wherein said cholinesterase inhibitor is rivastigmine.

7. The method of claim 6, wherein the amount of rivastigmine in the blood is less when compared to blood levels after oral delivery.

8. The method of claim 6, wherein the amount of rivastigmine delivered to the brain is higher than the amount delivered to the brain after oral administration.

9. The method of claim 1, wherein said patient has been diagnosed with Alzheimer's Disease.

10. The method of claim 1, further comprising c) monitoring said patient's symptom for a period of time of at least 2 weeks.

11. The method of claim 1, wherein said patient is a human patient.

12. The method of claim 1, wherein said position in the nose is the olfactory cleft.

13. The method of claim 1, wherein said position in the nose is the middle meatus.

14. A device, comprising an implant comprising a cholinesterase inhibitor.

15. The device of claim 14, wherein said implant comprises a polymer scaffold.

16. The device of claim 15, wherein said polymer scaffold comprises fibers.

17. The device of claim 14, wherein said implant releases said cholinesterase inhibitor by osmosis.

18. The device of claim 14, wherein said cholinesterase inhibitor is rivastigmine.
